(19) United States
(12) Patent Application Publication
(10) Pub. No.: US 2025/0171444 A1
(43) Pub. Date: May 29, 2025

(54) PI3Ka INHIBITORS

(71) Applicant: Synnovation Therapeutics, Inc., Wilmington, DE (US)

(72) Inventors: Chao Qi, Wilmington, DE (US); Jun Pan, Wilmington, DE (US); Jeffrey Yang, Wilmington, DE (US); Liangxing Wu, Wilmington, DE (US); Wenqing Yao, Wilmington, DE (US)

(21) Appl. No.: 18/843,508

(22) PCT Filed: Mar. 3, 2023
(86) PCT No.: PCT/US2023/063641

§ 371 Date: Sep. 3, 2024

Related U.S. Application Data
(60) Provisional application No. 63/316,437, filed on Mar. 4, 2022.

(51) Int. Cl. C07D 471/14; A61K 31/437; A61K 31/496; A61K 31/5377

(52) U.S. Cl. CPC C07D 471/14; A61K 31/437; A61K 31/496; A61K 31/5377

(57) ABSTRACT

The present disclosure provides compounds, compositions, and methods useful for inhibiting PI3Ka, and/or treating a disease, disorder, or condition associated with PI3Ka, and/or treating cancer. (Formula I)

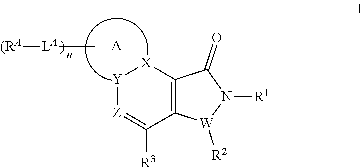

### TECHNICAL FIELD

The present disclosure provides heterocyclic compounds as well as their pharmaceutical compositions that modulate the activity of PI3Kα and are useful in the treatment of various diseases related to PI3Kα, including cancer.

### BACKGROUND

In the past few decades, signal transduction events have been studied to demonstrate critical roles in regulating almost all aspects of biological responses. Aberrant activation of the signaling pathways regulating cell survival and proliferation is commonly observed in many human cancers. The phosphoinositide 3-kinases (PI3Ks) signaling pathway is documented to be one of the highly mutated pathways in human cancers (Vogelstein et al., Science, 2013, 339(6127), 1546-1558). The PI3K signaling pathway regulates cell survival and proliferation. Increased activity of this pathway is associated with tumor progression and resistance to cancer therapies (Fusco et al., Front Oncol., 2021, 11, 644737).

PI3Ks belong to a lipid kinase family which catalyzes the phosphorylation of lipids contained in or associated with cell membranes. The PI3K family has fifteen kinases with distinct substrates, expression pattern, and modes of regulation. The class-I PI3Ks (p110α, p110β, p110δ, and p110γ) are typically activated by tyrosine receptor kinases or G-protein coupled receptors to generate PIP3, which activates downstream effectors of Akt, mTOR, or Rho GTPases (Fruman et al., Nat. Rev. Drug Discov., 2014, 13(2), 140-156).

Genetic mutations in the gene coding for PI3Kα are hotspot point mutations within helical and kinase domains, such as E542K, E545K and H1047R. These mutations have been observed to occur in many cancer types such as lung, stomach, endometrial, ovarian, bladder, breast, colon, brain, prostate, and skin cancers. Because these gain-of-function mutations in PI3Kα are associated with tumor progression, targeting this pathway may provide valuable therapeutic opportunities (Courtney et al., J. Clin. Oncol., 2010, 28 (6), 1075-1083). While multiple inhibitors of PI3Ks have been developed (for example, taselisib, alpelisib, buparlisib and others), these molecules inhibit multiple PI3K isoforms. These “pan-PI3K” inhibitors have encountered major hurdle in the clinical development due to inability to achieve the required level of target inhibition in tumors while avoiding toxicity in cancer patients (Fruman et al., Nat. Rev. Drug Discov., 2014, 13(2), 140-156). The toxicity of PI3K inhibitors is dependent on their isoform selectivity profile. Inhibition of PI3Kα is associated with hyperglycemia and rash, while inhibition of PI3K6 or PI3K7 is associated with diarrhea, myelosuppression, and transaminitis (Hanker et al., Cancer Discov., 2019, 9(4), 482-491). Therefore, selective inhibitors of PI3Kα may increase the therapeutic window, enabling sufficient target inhibition in the tumor while avoiding dose-limiting toxicity in cancer patients. However, given the central role of PI3Kα in regulating glucose homeostasis and other critical physiological process, current PI3Kα selective inhibitors, which are equally potent to wild-type and mutant PI3Kα, often cause hyperglycemia and/or hyperinsulinemia (Busaidy et al., J. Clin. Oncol., 2012, 30, 2919-2928). In summary, developing inhibitors with enhanced selectivity for mutant PI3Kα against wild-type PI3Kα would be able to overcome the problem of compensatory insulin production and hyperglycemia.

### SUMMARY

The present disclosure provides compounds and/or compositions useful for inhibiting PI3Kα. In some embodiments, provided compounds and/or compositions are useful for selectively inhibiting PI3Kα over other PI3K isoforms. In some embodiments, provided compounds and/or compositions are useful for selectively inhibiting mutant PI3Kα over wide-type PI3Kα. In some embodiments, provided compounds and/or compositions are useful, among other things, treating and/or preventing diseases, disorders, or conditions associated with PI3Kα. In some embodiments, provided compounds and/or compositions are useful, among other things, treating and/or preventing diseases, disorders, or conditions associated with mutant PI3Kα.

In some embodiments, the present disclosure provides certain compounds and/or compositions that are useful in medicine, and particularly for treating cancer.

In some embodiments, the present disclosure provides a compound of Formula I.

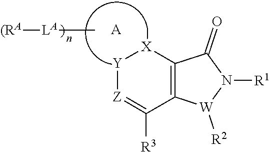

or a pharmaceutically acceptable salt thereof, wherein each of X, Y, Z, W, L^{A}, R^{A}, Ring A, R¹, R², R³ and n is as defined herein.

In some embodiments, provided compounds have structures of any of Formulae II, III and IV as described herein.

In some embodiments, the present disclosure provides compositions that comprise and/or deliver a provided compound. In some embodiments, such compositions are pharmaceutical compositions comprising a pharmaceutically acceptable carrier.

### DETAILED DESCRIPTION

### Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomic, and geometric (or conformational) mixtures of provided compounds are within the scope of the disclosure. For example, in some case, Table 1 shows one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. The isotopically-labeled compounds may have one or more atoms replaced by an atom having an atomic mass or mass number usually found in nature. Examples of isotopes present in compounds of the present disclosure include isotopes of hydrogen, carbon, nitrogen, oxygen, phosphorus, fluorine and chlorine, such as, but not limited to, ²H, ³H, ¹³C, ¹⁴C, ¹⁵N, ¹⁷O, ¹⁸O, ³⁵S and ¹⁸F. Certain isotopically-labeled compounds of the present disclosure, in addition to being useful as therapeutic agents, are also useful in drug and/or substrate tissue distribution assays, as analytical tools or as probes in other biological assays. In one aspect of the present disclosure, tritiated (e.g., ³H) and carbon-14 (e.g., ¹⁴C) isotopes are useful given their ease of detectability. In another aspect of the present invention, replacement of one or more hydrogen atoms with heavier isotopes such as deuterium, (e.g., ²H) can afford certain therapeutic advantages.

As used herein and unless otherwise specified, the suffix “-ene” is used to describe a bivalent group. Thus, any of the terms above can be modified with the suffix “-ene” to describe a bivalent version of that moiety. For example, a bivalent carbocycle is “carbocyclylene”, a bivalent aryl ring is “arylene”, a bivalent benzene ring is “phenylene”, a bivalent heterocycle is “heterocyclylene”, a bivalent heteroaryl ring is “heteroarylene”, a bivalent alkyl chain is “alkylene”, a bivalent alkenyl chain is “alkenylene”, a bivalent alkynyl chain is “alkynylene”, and so forth.

Aliphatic: As used herein, the term “aliphatic” refers to a straight-chain (i.e., unbranched) or branched, optionally substituted hydrocarbon chain that is completely saturated or that contains one or more units of unsaturation, or a monocyclic or bicyclic hydrocarbon that is completely saturated or that contains one or more units of unsaturation but which is not aromatic (also referred to herein as “carbocyclic” or “cycloaliphatic”), that, unless otherwise specified, has a single point of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, alkynyl groups and hybrids thereof. In some embodiments, “aliphatic” refers to a straight-chain (i.e., unbranched) or branched, optionally substituted hydrocarbon chain that is completely saturated or that contains one or more units of unsaturation that has a single point of attachment to the rest of the molecule.

Alkyl: The term “alkyl”, used alone or as part of a larger moiety, refers to a saturated optionally substituted straight or branched hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkenyl: The term “alkenyl”, used alone or as part of a larger moiety, refers to an optionally substituted straight or branched hydrocarbon chain having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Aryl: As used herein, the term “aryl” refers to monocyclic, bicyclic, and polycyclic ring systems having a total of six to fourteen ring members (e.g., C₆₋₁₄), wherein at least one ring in the system is aromatic and wherein each ring in the system contains three to seven ring members. The term “aryl” may be used interchangeably with the term “aryl ring”. In some embodiments, “aryl” refers to an aromatic ring system which includes, but not limited to, phenyl, naphthyl, anthracyl and the like, which may bear one or more substituents. Unless otherwise specified, “aryl” groups are hydrocarbons.

Bivalent: As used herein, the term “bivalent” refers to a chemical moiety with two points of attachment to the rest of the molecule. For example, “bivalent C₁₋₆ aliphatic,” refers to bivalent aliphatic groups that are as defined herein, containing 1-6 aliphatic carbon atoms.

Carbocyclyl: As used herein, the terms “carbocyclyl,” “carbocycle,” and “carbocyclic ring” refer to saturated or partially unsaturated cyclic aliphatic monocyclic, bicyclic, or polycyclic ring systems, as described herein, having from 3 to 14 members, wherein the aliphatic ring system is optionally substituted as described herein. Carbocyclic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, “carbocyclyl” (or “cycloaliphatic”) refers to an optionally substituted monocyclic C₃-C₈ hydrocarbon, or an optionally substituted C₅-C₁₀ bicyclic hydrocarbon that is completely saturated or that contains one or more units of unsaturation, but which is not aromatic, that has a single point of attachment to the rest of the molecule. The term “cycloalkyl” refers to an optionally substituted saturated ring system of about 3 to about 10 ring carbon atoms. In some embodiments, cycloalkyl groups have 3-6 carbons. Exemplary monocyclic cycloalkyl rings include cyclopropyl, cyclobutyl, cyclopentyl, cyclohexyl, and cycloheptyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Carrier: As used herein, the term “carrier” refers to a diluent, adjuvant, excipient, or vehicle with which a composition is administered. In some embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Heteroaryl: As used herein, the terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π electrons shared in a cyclic array; and having, in addition to carbon atoms, from one to five heteroatoms. Exemplary heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridonyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridinyl, thienopyrimidinyl, triazolopyridinyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, and benzoisoxazolyl. The term “heteroaryl” may be used interchangeably with the terms “heteroaryl ring”, “heteroaryl group”, or “heteroaromatic”, any of which terms include rings that are optionally substituted.

Heteroatom: As used herein, the term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, and “heterocyclic ring” are used interchangeably and refer to a stable 3- to 8-membered monocyclic or 5- to 10-membered bicyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl, heteroaryl, or cycloaliphatic rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)).

Partially Unsaturated: As used herein, the term “partially unsaturated”, when referring to a ring moiety, means a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: As used herein, the term “pharmaceutically acceptable salt” refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977).

Prevent or prevention: As used herein, the term “prevent” or “prevention,” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Substituted or optionally substituted: As described herein, compounds of this disclosure may contain “optionally substituted” moieties. In general, the term “substituted,” whether preceded by the term “optionally” or not, means that one or more hydrogens of the designated moiety are replaced with a suitable substituent (i.e., as described below for optionally substituted groups). “Substituted” applies to one or more hydrogens that are either explicit or implicit from the structure (e.g.,

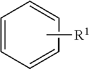

refers to at least

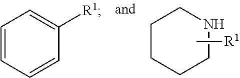

refers to at least

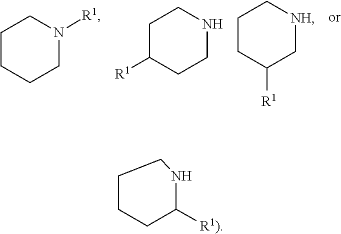

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄Rº; —(CH₂)₀₋₄ORº; —O(CH₂)₀₋₄Rº, —O—(CH₂)₀₋₄C(O)ORº; —(CH₂)₀₋₄CH(ORº)₂; —(CH₂)₀₋₄SRº; —(CH₂)₀₋₄Ph, which may be substituted with Rº; —(CH₂)₀₋₄O(CH₂)₀₋₁Ph which may be substituted with Rº; —CH═CHPh, which may be substituted with Rº; —(CH₂)₀₋₄O(CH₂)₀₋₁-pyridyl which may be substituted with Rº; —NO₂; —CN; —N₃; —(CH₂)₀₋₄N(Rº)₂; —(CH₂)₀₋₄N(Rº)C(O)Rº; —N(Rº)C(S)Rº; —(CH₂)₀₋₄N(Rº)C(O)NRº₂; —N(Rº)C(S)NRº₂; —(CH₂)₀₋₄N(Rº)C(O)ORº; —N(Rº)N(Rº)C(O)Rº; —N(Rº)N(Rº)C(O)NRº₂; —N(Rº)N(Rº)C(O)ORº; —(CH₂)₀₋₄C(O)Rº; —C(S)Rº; —(CH₂)₀₋₄C(O)ORº; —(CH₂)₀₋₄C(O)SRº; —(CH₂)₀₋₄C(O)OSiRº₃; —(CH₂)₀₋₄OC(O)Rº; —OC(O)(CH₂)₀₋₄SRº; —(CH₂)₀₋₄SC(O)Rº; —(CH₂)₀₋₄C(O)NRº₂; —C(S)NRº₂; —C(S)SRº; —SC(S)SRº, —(CH₂)₀₋₄OC(O)NRº₂; —C(O)N(ORº)Rº; —C(O)C(O)Rº; —C(O)CH₂C(O)Rº; —C(NORº)Rº; —(CH₂)₀₋₄SSRº; —(CH₂)₀₋₄S(O)₂Rº; —(CH₂)₀₋₄S(O)(═NRº)Rº; —(CH₂)₀₋₄S(O)₂ORº; —(CH₂)₀₋₄OS(O)₂Rº; —(CH₂)₀₋₄—S(O)₂NRº₂; —(CH₂)₀₋₄S(O)(═NRº)NRº₂; —(CH₂)₀₋₄S(O)Rº; —N(Rº)S(O)₂NRº₂; —N(Rº)S(O)₂Rº; —N(Rº)S(O)(═NRº)Rº; —N(ORº)Rº; —C(NH)NRº₂; —P(O)₂Rº; —P(O)Rº₂; —OP(O)Rº₂; —OP(O)(ORº)₂; —SiRº₃; —(C₁₋₄ straight or branched alkylene)O—N(Rº)₂; or —(C₁₋₄ straight or branched alkylene)C(O)O—N(Rº)₂, wherein each Rº may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of Rº, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on Rº (or the ring formed by taking two independent occurrences of Rº together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, —O(haloR^{●}), —(CH₂)₀₋₂CN, —N₃, —(CH₂)₀₋₂C(O)R^{●}, —(CH₂)₀₋₂C(O)OH, —(CH₂)₀₋₂C(O)OR^{●}, —(CH₂)₀₋₂C(O)NH₂, —(CH₂)₀₋₂C(O)NHR^{●}, —(CH₂)₀₋₂C(O)NR^{●}₂, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —(CH₂)₀₋₂NHC(O)R^{●}, —(CH₂)₀₋₂NR^{●}C(O)R^{●}, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of Rº include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R, —S(O)₂R, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{†}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Treat: As used herein, the term “treat” (also “treatment” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition.

### Provided Compounds

In some embodiments, the present disclosure provides a compound of Formula I:

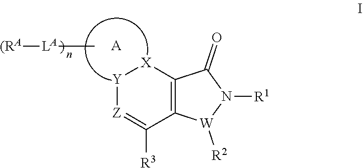

or a pharmaceutically acceptable salt thereof, wherein. X is N and Y is C; or X is C and Y is N;
Ring A is 5- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur;
each L^{A} is independently a covalent bond or optionally substituted bivalent C₁₋₆ aliphatic;
each R^{A} is independently oxo, halogen, —CN, —OR^{A1}, —SR^{A1}, —N(R^{A1})₂, —NO₂, —C(O)R^{A1}, —C(O)OR^{A1}, —C(O)N(R^{A1})₂, —C(O)NR^{A1}(OR^{A1}), —OC(O)R^{A1}, —OC(O)N(R^{A1})₂, —OC(O)OR^{A1}, —OSO₂R^{A1}, —OSO₂N(R^{A1})₂, —N(R^{A1})C(O)R^{A1}, —NR^{A1}C(O)OR^{A1}, —NR^{A1}C(O)N(R^{A1})₂, —N(R^{A1})SO₂R^{A1}, —NR^{A1}S(O)₂N(R^{A1})₂, —NR^{A1}OR^{A1}, —NR^{A1}S(O)R^{A1}, —NR^{A1}S(O)N(R^{A1})₂, —S(O)R^{A1}, —SO₂R^{A1}, —S(O)N(R^{A1})₂, —SO₂N(R^{A1})₂, —SO₃R^{A1}, —C(═NR^{m})R^{A1}, —C(═NR^{m})N(R^{A1})₂, —NR^{A1}C(═NR^{m})R^{A1}, —NR^{A1}C(═NR^{m})N(R^{A1})₂, —NR^{A1}S(O)(═NR^{m})R^{A1}, —NR^{A1}S(O)(═NR^{m})N(R^{A1})₂, —OS(O)(═NR^{m})R^{A1}, —S(O)(═NR^{m})R^{A1}, —S(O)(═NR^{m})N(R^{A1})₂, —P(O)(R^{A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
   wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents;
R¹ is hydrogen or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur;
W is CR^{W} or N;
R^{W} is hydrogen or -L^{W}-R^{2G};
L^{W} is a covalent bond or optionally substituted bivalent C₁₋₆ aliphatic;
R² is C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
   wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R² is independently optionally substituted with 1, 2, 3, 4, 5, or 6 R^{2G} substituents; or
   R² and R^{W} are taken together with the carbon atom to which they are attached to form an optionally substituted ring selected from 3- to 7-membered saturated or partially unsaturated monocyclic carbocyclyl, 5- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 5- to 10-membered saturated or partially unsaturated bicyclic heterocyclyl having 1-4 heteroatoms independently selected from nitrogen, oxygen, and sulfur;
R³ is -L³-R^{3A};
L³ is a covalent bond or optionally substituted bivalent C₁₋₆ aliphatic;
R^{3A} is hydrogen, halogen, —CN, —OR^{3A1}, —SR^{3A1}, —N(R^{3A1})₂—NO₂, —C(O)R^{3A1}, —C(O)OR^{3A1}, —C(O)N(R^{3A1})₂, —C(O)NR^{3A1}(OR^{3A1}), —OC(O)R^{3A1}, —OC(O)N(R^{3A1})₂, —OC(O)OR^{3A1}, —OSO₂R^{3A1}, —OSO₂N(R^{3A1})₂, —N(R^{3A1})C(O)R^{3A1}, —NR^{3A1}C(O)OR^{3A1}, —NR^{3A1}C(O)N(R^{3A1})₂—N(R^{3A1})SO₂R^{3A1}, —NR^{3A1}S(O)₂N(R^{3A1})₂, —NR^{3A1}OR^{3A1}, —NR^{3A1}S(O)R^{3A1}, —NR^{3A1}S(O)N(R^{3A})₂, —S(O)R^{3A1}, —SO₂R^{3A1}, —S(O)N(R^{3A1})₂, —SO₂N(R^{3A1})₂, —SO₃R^{3A1}, —C(═NR^{m})R^{3A1}, —C(═NR^{m})N(R^{3A1})₂, —NR^{3A1}C(═NR^{m})R^{3A1}, —NR^{3A1}C(═NR^{m})N(R^{3A1})₂, —NR^{3A1}S(O)(═NR^{m})R^{3A1}, —NR^{3A1}S(O)(═NR^{m})N(R^{3A1})₂, —OS(O)(═NR^{m})R^{3A1}, —S(O)(═NR^{m})R^{3A1}, —S(O)(═NR^{m})N(R^{3A1})₂, —P(O)(R^{3A})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
   wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{3A} is independently optionally substituted with 1, 2, 3, 4, 5, or 6 R^{3AG} substituents;
Z is N or CR^{Z};
R^{Z} is -L^{Z}-R^{ZA};
   L^{Z} is a covalent bond or optionally substituted bivalent C₁₋₆ aliphatic;
   R^{ZA} is hydrogen, halogen, —CN, —OR^{ZA1}, —SR^{ZA1}, —N(R^{ZA1})₂, —NO₂, —C(O)R^{ZA1}, —C(O)OR^{ZA1}, —C(O)N(R^{ZA1})₂, —C(O)NR^{ZA1}(OR^{ZA1}), —OC(O)R^{ZA1}, —OC(O)N(R^{ZA1})₂, —OC(O)OR^{ZA1}, —OSO₂R^{ZA1}, —OSO₂N(R^{ZA1})₂, —N(R^{ZA1})C(O)R^{ZA1}, —NR^{ZA1}C(O)OR^{ZA1}, —NR^{ZA1}C(O)N(R^{ZA1})₂, —N(R^{ZA1})SO₂R^{ZA1}, —NR^{ZA1}S(O)₂N(R^{ZA1})₂, —NR^{ZA1}OR^{ZA1}, —NR^{ZA1}S(O)R^{ZA1}, —NR^{ZA1}S(O)N(R^{ZA1})₂, —S(O)R^{ZA1}, —SO₂R^{ZA1}, —S(O)N(R^{ZA1})₂, —SO₂N(R^{ZA1})₂, —SO₃R^{ZA1}, —C(═NR^{m})R^{ZA1}, —C(═NR^{m})N(R^{ZA1})₂, —NR^{ZA1}C(═NR^{m})R^{ZA1}, —NR^{ZA1}C(═NR^{m})N(R^{ZA1})₂, —NR^{ZA1}S(O)(═NR^{m})R^{ZA1}, —NR^{ZA1}S(O)(═NR^{m})N(R^{ZA1})₂, —OS(O)(═NR^{m})R^{ZA1}, —S(O)(═NR^{m})R^{ZA1}, —S(O)(═NR^{m})N(R^{ZA1})₂, —P(O)(R^{ZA1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
      wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{ZA} is independently optionally substituted with 1, 2, 3, 4, 5, or 6 R^{ZAG} substituents;
R^{A1}, R^{3A1}, and R^{ZA1} are each independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
   wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A1}, R^{3A1}, or R^{ZA1} is independently optionally substituted with 1, 2, 3, 4, 5, or 6 R^{G1} substituents;
   or two R^{A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
   or two R^{3A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
   or two R^{ZA1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
R^{AG}, R^{2G}, R^{3AG}, R^{ZAG}, and R^{G1} are each independently hydrogen, halogen, —CN, —OR, —SR, —N(R)₂, —NO₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
each R is independently hydrogen or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or
   two R when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
each R^{m} is independently —OH, —CN, or R; and
n is 0, 1, 2, 3, 4, or 5.

In some embodiments, the present disclosure provides a compound of Formula II:

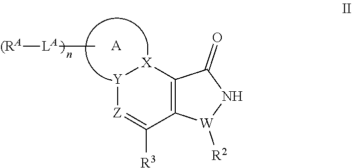

or a pharmaceutically acceptable salt thereof, wherein each of X, Y, Z, W, L^{A}, R^{A}, Ring A, R², R³ and n is as defined above for Formula I and described in classes and subclasses herein, both singly and in combination.

In some embodiments, the present disclosure provides a compound of Formula III:

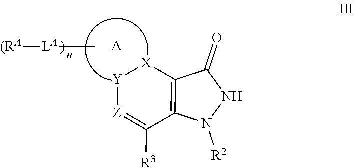

or a pharmaceutically acceptable salt thereof, wherein each of X, Y, Z, L^{A}, R^{A}, Ring A, R², R³ and n is as defined above for Formula I and described in classes and subclasses herein, both singly and in combination.

In some embodiments, the present disclosure provides a compound of Formula IV:

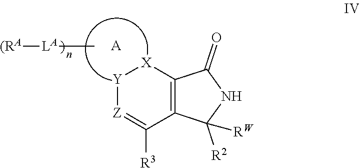

or a pharmaceutically acceptable salt thereof, wherein each of X, Y, Z, L^{A}, R^{A}, Ring A, R^{W}, R², R³ and n is as defined above for Formula I and described in classes and subclasses herein, both singly and in combination.

In some embodiments of any of Formulae I, II, III and IV, X is C and Y is N.

In some embodiments of any of Formulae I, II, III and IV, X is N and Y is C.

As described above, in some embodiments of any of Formulae I, II, III and IV, Ring A is 5- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur.

In some embodiments, Ring A is 5- to 6-membered monocyclic heteroaryl having 1-4 heteroatoms independently selected from nitrogen, oxygen, and sulfur. In some embodiments, Ring A is 5-membered monocyclic heteroaryl having 1-4 heteroatoms independently selected from nitrogen, oxygen, and sulfur. In some embodiments, Ring A is 5-membered monocyclic heteroaryl having 1-4 nitrogen atoms. In some embodiments, Ring A is 5-membered monocyclic heteroaryl having 1-3 nitrogen atoms. In some embodiments, Ring A is 5-membered monocyclic heteroaryl having 1-2 nitrogen atoms. In some embodiments, Ring A is 5-membered monocyclic heteroaryl having 1 nitrogen atom. In some embodiments, Ring A is pyrrolyl, pyrazolyl, imidazolyl or triazolyl.

As described above, in some embodiments of any of Formulae I, II, III and IV, each L^{A} is independently a covalent bond or optionally substituted bivalent C₁₋₆ aliphatic. In some embodiments, each L^{A} is a covalent bond.

As described above, in some embodiments of any of Formulae I, II, III and IV, each R^{A} is independently oxo, halogen, —CN, —OR^{A1}, —SR^{A1}, —N(R^{A1})₂, —NO₂, —C(O)R^{A1}, —C(O)OR^{A1}, —C(O)N(R^{A1})₂, —C(O)NR^{A1}(OR^{A1}), —OC(O)R^{A1}, —OC(O)N(R^{A1})₂, —OC(O)OR^{A1}, —OSO₂R^{A1}, —OSO₂N(R^{A1})₂, —N(R^{A1})C(O)R^{A1}, —NR^{A1}C(O)OR^{A1}, —NR^{A1}C(O)N(R^{A1})₂, —N(R^{A1})SO₂R^{A1}, —NR^{A1}S(O)₂N(R^{A1})₂, —NR^{A1}OR^{A1}, —NR^{A1}S(O)R^{A1}, —NR^{A1}S(O)N(R^{A1})₂, —S(O)R^{A1}, —SO₂R^{A1}, —S(O)N(R^{A1})₂, —SO₂N(R^{A1})₂, —SO₃R^{A1}, —C(═NR^{m})R^{A1}, —C(═NR^{m})N(R^{A1})₂—NR^{A1}C(═NR^{m})R^{A1}, —NR^{A1}C(═NR^{m})N(R^{A1})₂, —NR^{A1}S(O)(═NR^{m})R^{A1}, —NR^{A1}S(O)(═NR^{m})N(R^{A1})₂, —OS(O)(═NR^{m})R^{A1}, —S(O)(═NR^{m})R^{A1}, —S(O)(═NR^{m})N(R^{A1})₂, —P(O)(R^{A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents.

In some embodiments, a single instance of R^{A} is independently —C(O)OR^{A1}.

In some embodiments of any of Formulae I, II, III and IV, each R^{A1} is independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A1} is independently optionally substituted with 1, 2, 3, 4, 5, or 6 R^{G1} substituents; or two R^{A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

In some embodiments, each R^{A1} is independently C₁₋₆ aliphatic. In some embodiments, each R^{A1} is independently C₁₋₃ aliphatic. In some embodiments, each R^{A1} is independently C₁₋₂ aliphatic. In some embodiments, a single instance of R^{A1} is ethyl.

In some embodiments of any of Formulae I, II, III and IV, each R^{AG} is independently hydrogen, halogen, —CN, —OR, —SR, —N(R)₂, —NO₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments,

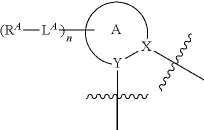

is selected from the group consisting

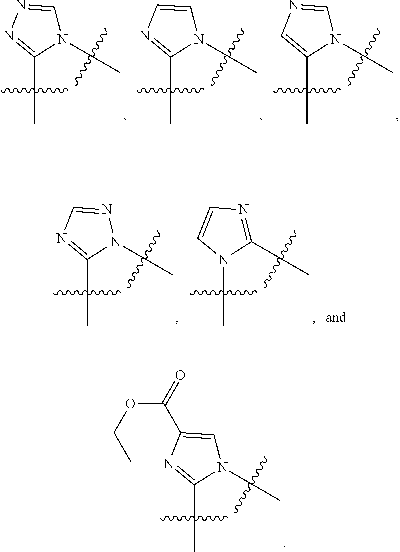 [SMILES: *c1nncn1* ; *c1nccn1* ; *c1cncn1* ; *c1ncnn1* ; *c1nccn1* ; *c1nc(C(=O)OCC)cn1*]

As described above, in some embodiments of Formula I, R¹ is hydrogen or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur.

In some embodiments, R¹ is hydrogen.

As described above, in some embodiments of any of Formulae I and II, W is CR^{W} or N. In some embodiments, W is CR^{W}. In some embodiments, W is N.

As described above, in some embodiments of any of Formulae I, II, and IV, R^{W} is hydrogen or -L^{W}-R^{2G}. In some embodiments, R^{W} is hydrogen.

As described above, in some embodiments of any of Formulae I, II, and IV, L^{W} is a covalent bond or optionally substituted bivalent C₁₋₆ aliphatic. In some embodiments, L^{W} is a covalent bond.

As described above, in some embodiments of any of Formulae I, II, III and IV, R² is C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R² is independently optionally substituted with 1, 2, 3, 4, 5, or 6 R^{2G} substituents; or R² and R^{W} are taken together with the carbon atom to which they are attached to form an optionally substituted ring selected from 3- to 7-membered saturated or partially unsaturated monocyclic carbocyclyl, 5- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 5- to 10-membered saturated or partially unsaturated bicyclic heterocyclyl having 1-4 heteroatoms independently selected from nitrogen, oxygen, and sulfur.

In some embodiments, R² is phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, each of which is optionally substituted with 1, 2, 3, 4, 5, or 6 independently selected R^{2G} substituents.

In some embodiments, R² is phenyl optionally substituted with 1, 2, 3, 4, 5, or 6 R^{2G} substituents. In some embodiments, R² is phenyl optionally substituted with 1, 2, or 3 R^{2G} substituents. In some embodiments, R² is phenyl optionally substituted with 1 or 2 R^{2G} substituents. In some embodiments, R² is phenyl optionally substituted with 2 R^{2G} substituents. In some embodiments, R² is phenyl substituted with 1, 2, 3, 4, 5, or 6 R^{2G} substituents. In some embodiments, R² is phenyl substituted with 1, 2, or 3 R^{2G} substituents. In some embodiments, R² is phenyl substituted with 1 or 2 R^{2G} substituents. In some embodiments, R² is phenyl substituted with 2 R^{2G} substituents.

In some embodiments of any of Formulae I, II, III and IV, each R^{2G} is independently hydrogen, halogen, —CN, —OR, —SR, —N(R)₂, —NO₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments, each R^{2G} is hydrogen. In some embodiments, each R^{2G} is independently halogen. In some embodiments, each R^{2G} is independently —F, —Cl, or —Br. In some embodiments, each R^{2G} is independently —F or —Cl. In some embodiments, a single instance of R^{2G} is —F. In some embodiments, a single instance of R^{2G} is —Cl.

In some embodiments, R² is

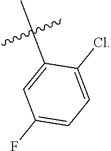 [SMILES: *c1cc(F)ccc1Cl]

As described above, in some embodiments of any of Formulae I, II, III and IV, R³ is -L³-R^{3A}.

As described above, in some embodiments of any of Formulae I, II, III and IV, L³ is a covalent bond or optionally substituted bivalent C₁₋₆ aliphatic.

In some embodiments, L³ is a covalent bond.

As described above, in some embodiments of any of Formulae I, II, III and IV, R^{3A} is hydrogen, halogen, —CN, —OR^{3A1}, —SR^{3A1}, —N(R^{3A1})₂, —NO₂, —C(O)R^{3A1}, —C(O)OR^{3A1}, —C(O)N(R^{3A1})₂, —C(O)NR^{3A1}(OR^{3A1}), —OC(O)R^{3A1}, —OC(O)N(R^{3A1})₂, —OC(O)OR^{3A1}, —OSO₂R^{3A1}, —OSO₂N(R^{3A1})₂, —N(R^{3A1})C(O)R^{3A1}, —NR^{3A1}C(O)OR^{3A1}, —NR^{3A1}C(O)N(R^{3A1})₂—N(R^{3A1})SO₂R^{3A1}, —NR^{3A1}S(O)₂N(R^{3A1})₂, —NR^{3A1}OR^{3A1}, —NR^{3A1}S(O)R^{3A1}, —NR^{3A1}S(O)N(R^{3A1})₂, —S(O)R^{3A1}, —SO₂R^{3A1}, —S(O)N(R^{3A1})₂, —SO₂N(R^{3A1})₂, —SO₃R^{3A1}, —C(═NR^{m})R^{3A1}, —C(═NR^{m})N(R^{3A1})₂, —NR^{3A1}C(═NR^{m})R^{3A1}, —NR^{3A1}C(═NR^{m})N(R^{3A1})₂, —NR^{3A1}S(O)(═NR^{m})R^{3A1}, —NR^{3A1}S(O)(═NR^{m})N(R^{3A1})₂, —OS(O)(═NR^{m})R^{3A1}, —S(O)(═NR^{m})R^{3A1}, —S(O)(═NR^{m})N(R^{3A1})₂, —P(O)(R^{3A})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{3A} is independently optionally substituted with 1, 2, 3, 4, 5, or 6 R^{3AG} substituents.

In some embodiments, R^{3A} is —N(R^{3A1})C(O)R^{3A1} or —NR^{3A1}C(O)N(R^{3A1})₂. In some embodiments, R^{3A} is —N(R^{3A1})C(O)R^{3A1}. In some embodiments, R^{3A} is —NR^{3A1}C(O)N(R^{3A1})₂. In some embodiments, R^{3A} is —NHC(O)R^{3A1} or —NHC(O)N(R^{3A1})₂. In some embodiments, R^{3A} is —NHC(O)R^{3A1}. In some embodiments, R^{3A} is —NHC(O)N(R^{3A1})₂.

In some embodiments of any of Formulae I, II, III and IV, each R^{3A1} is independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{3A1} is independently optionally substituted with 1, 2, 3, 4, 5, or 6 R^{G1} substituents; or two R^{3A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

In some embodiments, each R^{3A1} is independently hydrogen or 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of carbocyclyl, phenyl, heterocyclyl and heteroaryl is independently optionally substituted with 1, 2, 3, 4, 5, or 6 R^{G1} substituents. In some embodiments, each R^{3A1} is independently hydrogen or phenyl optionally substituted with 1, 2, 3, 4, 5, or 6 R^{G1} substituents.

In some embodiments, each R^{3A1} is hydrogen. In some embodiments, each R^{3A1} is independently 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, each of which is independently optionally substituted with 1, 2, 3, 4, 5, or 6 R^{G1} substituents;

In some embodiments, each R^{3A1} is independently phenyl optionally substituted with 1, 2, 3, 4, 5, or 6 R^{G1} substituents. In some embodiments, each R^{3A1} is independently phenyl optionally substituted with 1, 2, or 3 R^{G1} substituents. In some embodiments, each R^{3A1} is independently phenyl optionally substituted with 1 or 2 R^{G1} substituents. In some embodiments, each R^{3A1} is independently phenyl optionally substituted with 2 R^{G1} substituents. In some embodiments, each R^{3A1} is independently phenyl substituted with 1, 2, 3, 4, 5, or 6 R^{G1} substituents. In some embodiments, a single instance of R^{3A1} is independently phenyl substituted with 1, 2, or 3 R^{G1} substituents. In some embodiments, a single instance of R^{3A1} is independently phenyl substituted with 1 or 2 R^{G1} substituents. In some embodiments, a single instance of R^{3A1} is independently phenyl substituted with 2 R^{G1} substituents.

In some embodiments of any of Formulae I, II, III and IV, each R^{3AG} is independently hydrogen, halogen, —CN, —OR, —SR, —N(R)₂, —NO₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments, R³ is

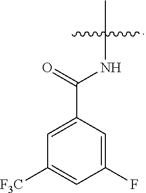 [SMILES: *NC(=O)c1cc(F)cc(C(F)(F)F)c1]

As described above, in some embodiments of any of Formulae I, II, III and IV, Z is N or CR^{Z}. In some embodiments, Z is N. In some embodiments, Z is CR^{Z}.

As described above, in some embodiments of any of Formulae I, II, III and IV, R^{Z} is -L^{Z}-R^{ZA}.

As described above, in some embodiments of any of Formulae I, II, III and IV, L^{Z} is a covalent bond or optionally substituted bivalent C₁₋₆ aliphatic. In some embodiments, L^{Z} is a covalent bond.

As described above, in some embodiments of any of Formulae I, II, III and IV, R^{ZA} is hydrogen, halogen, —CN, —OR^{ZA1}, —SR^{ZA1}, —N(R^{ZA1})₂, —NO₂, —C(O)R^{ZA1}, —C(O)OR^{ZA1}, —C(O)N(R^{ZA1})₂, —C(O)NR^{ZA1}(OR^{ZA1}), —OC(O)R^{ZA1}, —OC(O)N(R^{ZA1})₂, —OC(O)OR^{ZA1}, —OSO₂R^{ZA1}, —OSO₂N(R^{ZA1})₂, —N(R^{ZA1})C(O)R^{ZA1}, —NR^{ZA1}C(O)OR^{ZA1}, —NR^{ZA1}C(O)N(R^{ZA1})₂—N(R^{ZA1})SO₂R^{ZA1}, —NR^{ZA1}S(O)₂N(R^{ZA1})₂, —NR^{ZA1}OR^{ZA1}, —NR^{ZA1}S(O)R^{ZA1}, —NR^{ZA1}S(O)N(R^{ZA1})₂, —S(O)R^{ZA1}, —SO₂R^{ZA1}, —S(O)N(R^{ZA1})₂, —SO₂N(R^{ZA1})₂, —SO₃R^{ZA1}, —C(═NR^{m})R^{ZA1}, —C(═NR^{m})N(R^{ZA1})₂, —R^{ZA1}C(═NR^{m})R^{ZA1}, —NR^{ZA1}C(═NR^{m})N(R^{ZA1})₂, —NR^{ZA1}S(O)(═NR^{m})R^{ZA1}, —NR^{ZA1}S(O)(═NR^{m})N(R^{ZA1})₂, —OS(O)(═NR^{m})R^{ZA1}, —S(O)(═NR^{m})R^{ZA1}, —S(O)(═NR^{m})N(R^{ZA1})₂, —P(O)(R^{ZA1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{ZA} is independently optionally substituted with 1, 2, 3, 4, 5, or 6 R^{ZAG} substituents.

In some embodiments, R^{ZA} is hydrogen.

In some embodiments of any of Formulae I, II, III and IV, each R^{ZA1} is independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{ZA1} is independently optionally substituted with 1, 2, 3, 4, 5, or 6 R^{G1} substituents; or two R^{ZA1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

In some embodiments of any of Formulae I, II, III and IV, each R^{ZAG} is independently hydrogen, halogen, —CN, —OR, —SR, —N(R)₂, —NO₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments of any of Formulae I, II, III and IV, each R^{G1} is independently hydrogen, halogen, —CN, —OR, —SR, —N(R)₂, —NO₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments, each R^{G1} is independently halogen or optionally substituted C₁₋₆ aliphatic. In some embodiments, each R^{G1} is independently halogen. In some embodiments, each R^{G1} is independently —F, —Cl, or —Br. In some embodiments, a single instance of R^{G1} is —F.

In some embodiments, each R^{G1} is independently optionally substituted C₁₋₆ aliphatic. In some embodiments, each R^{G1} is independently optionally substituted C₁₋₃ aliphatic. In some embodiments, each R^{G1} is independently optionally substituted C₁₋₂ aliphatic. In some embodiments, each R^{G1} is independently optionally substituted methyl. In some embodiments, each R^{G1} is independently C₁₋₂ aliphatic optionally substituted with 1-5 halogen atoms. In some embodiments, each R^{G1} is independently C₁₋₂ aliphatic optionally substituted with 1-3 halogen atoms. In some embodiments, a single instance of R^{G1} is —CF₃.

As described above, in some embodiments of any of Formulae I, II, III and IV, each R is independently hydrogen or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or two R when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur.

As described above, in some embodiments of any of Formulae I, II, III and IV, each R^{m} is independently —OH, —CN, or R.

As described above, in some embodiments of any of Formulae I, II, III and IV, n is 0, 1, 2, 3, 4, or 5. In some embodiments, n is 0. In some embodiments, n is 1, 2, 3, 4, or 5. In some embodiments, n is 1.

In some embodiments, the present disclosure provides compounds selected from Table 1, or a pharmaceutically acceptable salt thereof.

TABLE 1

| | |
|---|---|
| 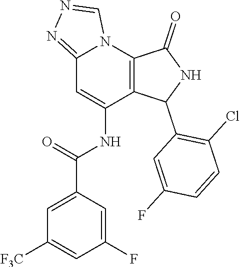 [SMILES: O=C(Nc1cc2nncn2c2c1C(c1cc(F)ccc1Cl)NC2=O)c1cc(F)cc(C(F)(F)F)c1] | I-1 |
| 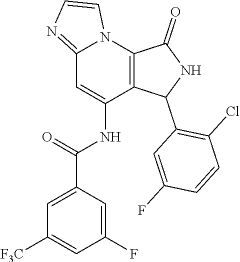 [SMILES: O=C(Nc1cc2nccn2c2c1C(c1cc(F)ccc1Cl)NC2=O)c1cc(F)cc(C(F)(F)F)c1] | I-2 |
| 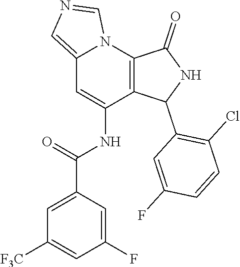 [SMILES: O=C(Nc1cc2cncn2c2c1C(c1cc(F)ccc1Cl)NC2=O)c1cc(F)cc(C(F)(F)F)c1] | I-3 |
| 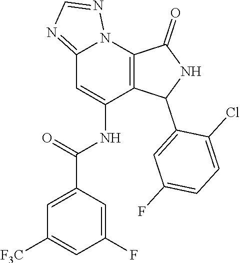 [SMILES: O=C(Nc1cc2ncnn2c2c1C(c1cc(F)ccc1Cl)NC2=O)c1cc(F)cc(C(F)(F)F)c1] | I-4 |
| 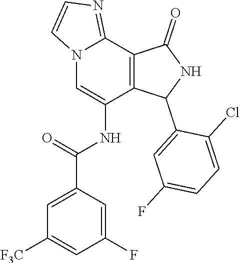 [SMILES: O=C(Nc1cn2ccnc2c2c1C(c1cc(F)ccc1Cl)NC2=O)c1cc(F)cc(C(F)(F)F)c1] | I-5 |
| 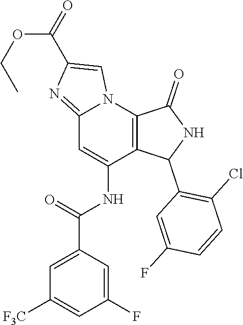 [SMILES: CCOC(=O)c1cn2c3c(c(NC(=O)c4cc(F)cc(C(F)(F)F)c4)cc2n1)C(c1cc(F)ccc1Cl)NC3=O] | I-6 |

In some embodiments, the compound provided herein is selected from:

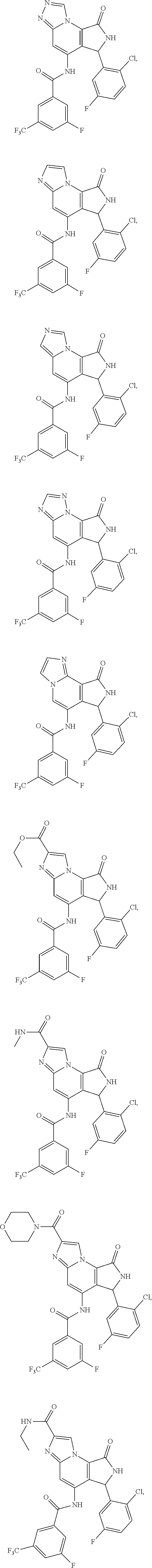

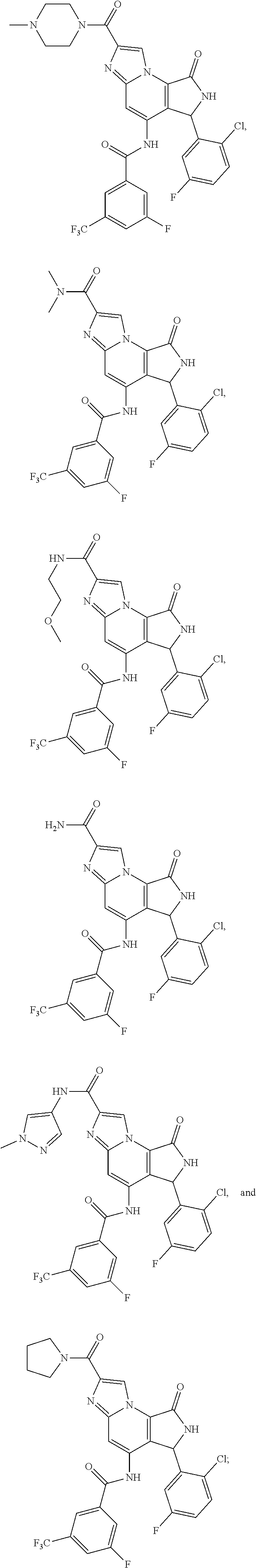 [SMILES: CN1CCN(C(=O)c2cn3c4c(c(NC(=O)c5cc(F)cc(C(F)(F)F)c5)cc3n2)C(c2cc(F)ccc2Cl)NC4=O)CC1 ; CN(C)C(=O)c1cn2c3c(c(NC(=O)c4cc(F)cc(C(F)(F)F)c4)cc2n1)C(c1cc(F)ccc1Cl)NC3=O ; COCCNC(=O)c1cn2c3c(c(NC(=O)c4cc(F)cc(C(F)(F)F)c4)cc2n1)C(c1cc(F)ccc1Cl)NC3=O ; NC(=O)c1cn2c3c(c(NC(=O)c4cc(F)cc(C(F)(F)F)c4)cc2n1)C(c1cc(F)ccc1Cl)NC3=O ; Cn1cc(NC(=O)c2cn3c4c(c(NC(=O)c5cc(F)cc(C(F)(F)F)c5)cc3n2)C(c2cc(F)ccc2Cl)NC4=O)cn1 ; O=C(Nc1cc2nc(C(=O)N3CCCC3)cn2c2c1C(c1cc(F)ccc1Cl)NC2=O)c1cc(F)cc(C(F)(F)F)c1]

or a pharmaceutically acceptable salt thereof.

In some embodiments, the present disclosure encompasses the recognition that provided compounds display certain desirable characteristics, e.g., as compared to other known compounds. For example, in some embodiments, provided compounds are more potent in one or more biochemical or cellular assays described herein, and/or have one or more other characteristics that make them more suitable for drug development, such as better selectivity for mutant PI3Kα over wide-type (WT) PI3Kα and/or better ADME (absorption, distribution, metabolism, and excretion) properties including but not limited to better permeability, cytotoxicity, hepatocyte stability, solubility, and/or plasma protein binding profiles, than other known compounds. In some embodiments, provided compounds display certain desirable characteristics in one or more assays described herein, e.g., compared to other known compounds.

In some embodiments, provided compounds are provided and/or utilized in a salt form (e.g., a pharmaceutically acceptable salt form). Reference to a compound provided herein is understood to include reference to salts thereof, unless otherwise indicated.

It will be understood that, unless otherwise specified or prohibited by the foregoing definition of any of Formulae I, II, III, and IV, embodiments of variables X, Y, Ring A, L^{A}R^{A}, R^{A1}, R^{AG}, R¹, W, R^{W}, L^{W}, R^{2G}, R², R³, L³, R^{3A}, R^{3A1}, R^{3AG}, Z, R^{Z}, L^{Z}, R^{ZA}, R^{ZA1}, R^{ZAG}, R^{G1}, R, R^{m}, and n, as defined above and described in classes and subclasses herein, apply to compounds of any of Formulae I, II, III and IV, both singly and in combination.

It will be appreciated that throughout the present disclosure, unless otherwise indicated, reference to a compound of Formula I is intended to also include any of Formulae I, II, III, and IV, and compound species of such formulae disclosed herein.

### Preparing Provided Compounds

Compounds of the present disclosure, including salts thereof, can be prepared using known organic synthesis techniques and can be synthesized according to numerous possible synthetic routes, such as those in the schemes below. The schemes below provide general guidance in connection with preparing the compounds of the present disclosure. One skilled in the art would understand that the preparations shown in the schemes can be modified or optimized using general knowledge of organic chemistry to prepare various compounds of the present disclosure. Provided compounds may generally be made by the processes described in the ensuing schemes and examples.

In some embodiments, provided compounds are prepared according to the following Scheme:

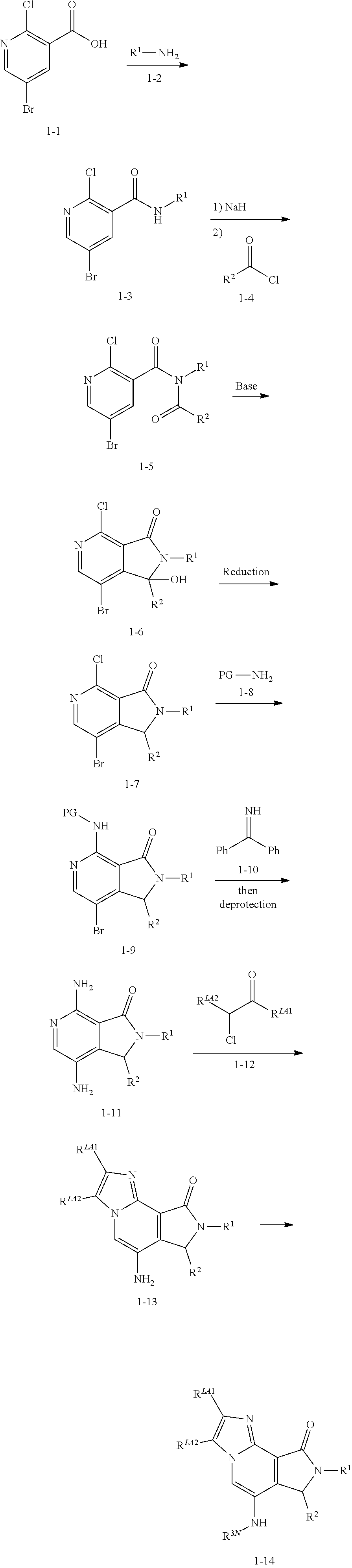

wherein PG is a suitable protecting group (e.g., 2,4-dimethocybenzyl), R^{LA1} and R^{LA2} are each independently -L^{A}-R^{A}, R^{3N} is R^{3A1}, COR^{3A1} or CON(R^{3A1})₂, and each of L^{A}, R^{A}, R¹, R², and R^{3A1} is as defined above for Formula I, and described in classes and subclasses herein, both singly and in combination. Accordingly, in some embodiments, intermediate 1-3 is prepared by a process comprising contacting compounds of Formulae 1-1 and 1-2. In some embodiments, intermediate 1-5 is prepared by a process comprising contacting intermediate 1-3 with a suitable base (e.g., NaH) and compounds of Formula 1-4. In some embodiments, intermediate 1-6 is prepared by a process comprising contacting intermediate 1-5 with a suitable base (e.g., LiHMDS). In some embodiments, intermediate 1-7 is prepared by a process comprising reacting intermediate 1-6 under suitable conditions (e.g., Et₃SiH/TFA). In some embodiments, intermediate 1-9 is prepared by a process comprising contacting intermediate 1-7 with compounds of Formula 1-8 (e.g., 2,4-dimethoxybenzylamine). In some embodiments, intermediate 1-11 is prepared by a process comprising contacting intermediate 1-9 with compounds of Formula 1-10 under suitable conditions (e.g., transition metal-catalyzed cross-coupling reaction conditions such as Buchwald-Hartwig cross-coupling reaction conditions) followed by removal of the protecting group. In some embodiments, intermediate 1-13 is prepared by a process comprising contacting intermediate 1-11 with compounds of Formula 1-12. In some embodiments, compounds of Formula 1-14 are prepared by a process comprising reacting intermediate 1-13 under suitable conditions (e.g., nucleophilic aromatic substitution, nucleophilic addition, amide coupling, or transition metal-catalyzed cross-coupling reaction conditions).

In some embodiments, provided compounds are prepared according to the following Scheme:

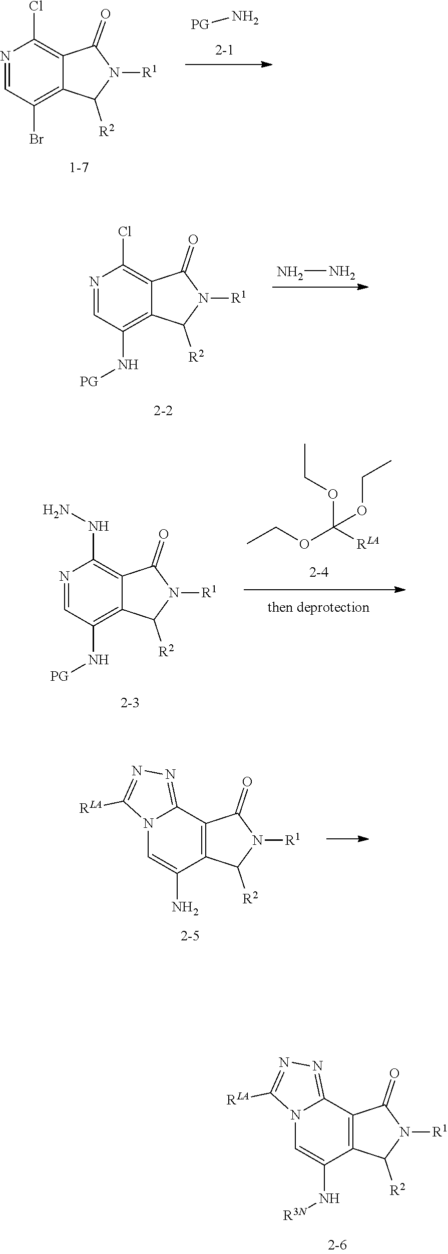

wherein PG is a suitable protecting group (e.g., 2,4-dimethocybenzyl), R^{LA} is -L^{A}-R^{A}, R^{3N} is R^{3A1}, COR^{3A1} or CON(R^{3A1})₂, and each of L^{A}, R^{A}, R¹, R², and R^{3A1} is as defined above for Formula I, and described in classes and subclasses herein, both singly and in combination. Accordingly, in some embodiments, intermediate 2-2 is prepared by a process comprising contacting compounds of Formulae 1-7 and 2-1 under suitable conditions (e.g., transition metal-catalyzed cross-coupling reaction conditions). In some embodiments, intermediate 2-3 is prepared by a process comprising contacting intermediate 2-2 with hydrazine. In some embodiments, intermediate 2-5 is prepared by a process comprising contacting intermediate 2-3 with compounds of Formula 2-4 followed by removal of the protecting group. In some embodiments, compounds of Formula 2-6 are prepared by a process comprising reacting intermediate 2-5 under suitable conditions (e.g., nucleophilic aromatic substitution, nucleophilic addition, amide coupling, or transition metal-catalyzed cross-coupling reaction conditions).

In some embodiments, provided compounds are prepared according to the following Scheme:

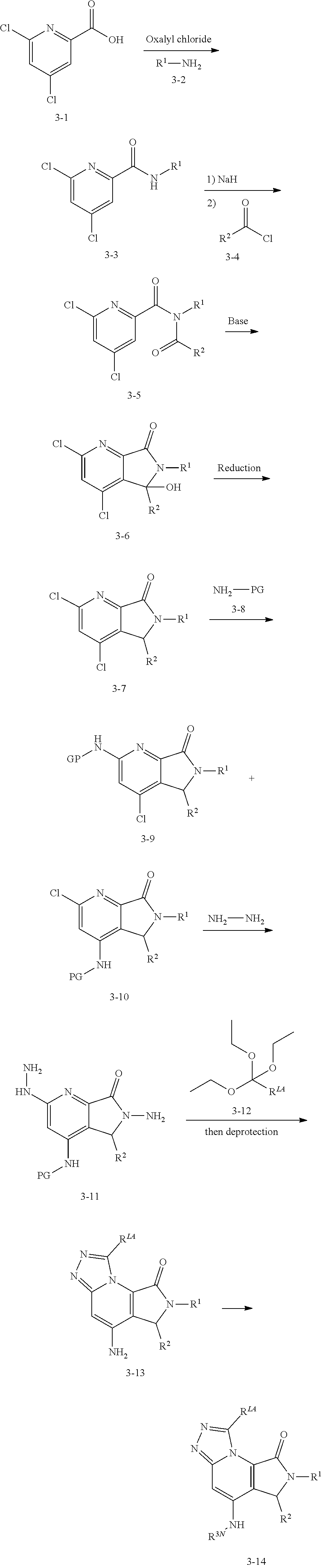

wherein PG is a suitable protecting group (e.g., 2,4-dimethocybenzyl), R^{LA} is -L^{A}-R^{A}, R^{3N} is R^{3A1}, COR^{3A1} or CON(R^{3A1})₂, and each of L^{A}, R^{A}, R¹, R², and R^{3A1} is as defined above for Formula I, and described in classes and subclasses herein, both singly and in combination. Accordingly, in some embodiments, intermediate 3-3 is prepared by a process comprising contacting compounds of Formulae 3-1 with oxalyl chloride and compounds of Formula 3-2. In some embodiments, intermediate 3-5 is prepared by a process comprising contacting intermediate 3-3 with a suitable base (e.g., NaH) and compounds of Formula 3-4. In some embodiments, intermediate 3-6 is prepared by a process comprising contacting intermediate 3-5 with a suitable base (e.g., LiHMDS). In some embodiments, intermediate 3-7 is prepared by a process comprising reacting intermediate 3-6 under suitable conditions (e.g., Et₃SiH/TFA, BF₃·OEt/Et₃SiH). In some embodiments, intermediates 3-9 and 3-10 are prepared by a process comprising contacting intermediate 3-7 with compounds of Formula 3-8 (e.g., 2,4-dimethoxybenzylamine). In some embodiments, intermediate 3-11 is prepared by a process comprising contacting intermediates 3-9 and 3-10 with hydrazine monohydrate. In some embodiments, intermediate 3-13 is prepared by a process comprising contacting intermediate 3-11 with compounds of Formula 3-12 followed by removal of the protecting group. In some embodiments, compounds of Formula 3-14 are prepared by a process comprising reacting intermediate 3-13 under suitable conditions (e.g., nucleophilic aromatic substitution, nucleophilic addition, amide coupling, or transition metal-catalyzed cross-coupling reaction conditions).

In some embodiments, provided compounds are prepared according to the following Scheme:

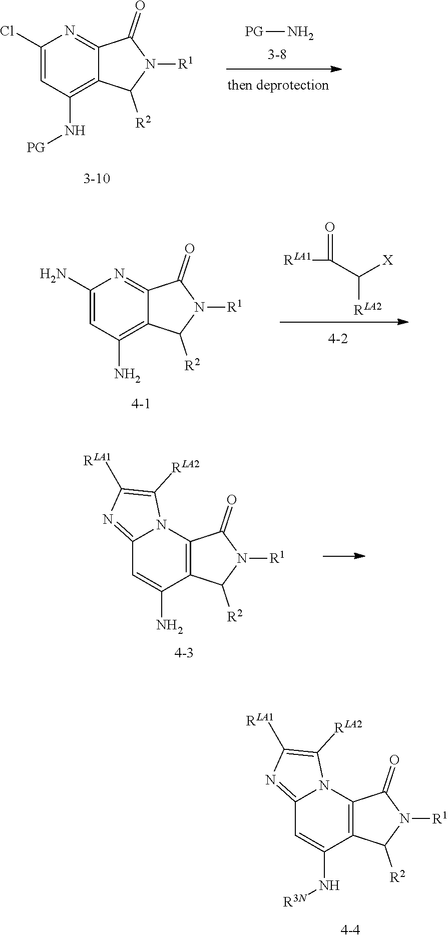

wherein PG is a suitable protecting group (e.g., 2,4-dimethocybenzyl), R^{LA1} and R^{LA2} are each independently -L^{A}-R^{A}, R^{3N} is R^{3A1}, COR^{3A1} or CON(R^{3A1})₂, and each of L^{A}, R^{A}, R¹, R², and R^{3A1} is as defined above for Formula I, and described in classes and subclasses herein, both singly and in combination. Accordingly, in some embodiments, intermediate 4-1 is prepared by a process comprising contacting compounds of Formulae 3-10 and 3-8 (e.g., 2,4-dimethoxybenzylamine) under suitable conditions (e.g., transition metal-catalyzed cross-coupling reaction conditions) followed by removal of the protecting group. In some embodiments, intermediate 4-3 is prepared by a process comprising contacting intermediate 4-1 with compounds of 4-2. In some embodiments, compounds of Formula 4-4 are prepared by a process comprising reacting intermediate 4-3 under suitable conditions (e.g., nucleophilic aromatic substitution, nucleophilic addition, amide coupling, or transition metal-catalyzed cross-coupling reaction conditions).

In some embodiments, provided compounds are prepared according to the following Scheme:

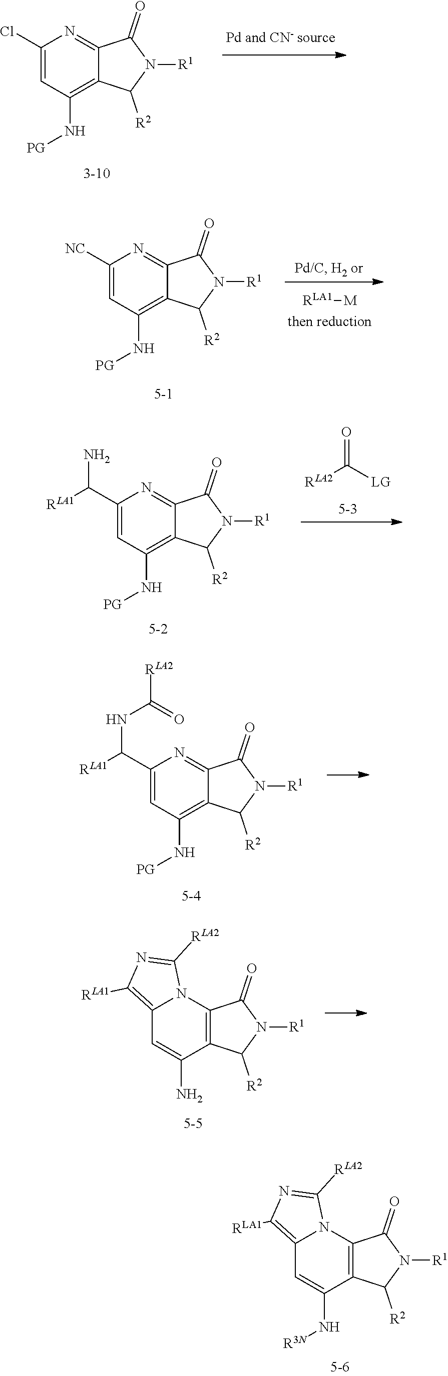

wherein PG is a suitable protecting group (e.g., 2,4-dimethocybenzyl), LG is a suitable leaving group such as Cl or OH, R^{LA1} and R^{LA} are each independently -L^{A}-R^{A}, R^{3N} is R^{3A1}, COR^{3A1} or CON(R^{3A1})₂, and each of L^{A}, R^{A}, R¹, R², and R^{3A1} is as defined above for Formula I, and described in classes and subclasses herein, both singly and in combination. Accordingly, in some embodiments, intermediate 5-1 is prepared by a process comprising reacting compounds of Formula 3-10 in the presence of a suitable reagent comprising Pd and a suitable reagent comprising —CN. In some embodiments, intermediate 5-2 is prepared by a process comprising reacting intermediate 5-1 under suitable conditions (e.g., hydrogenation conditions) or contacting intermediate 5-1 with a suitable reagent (e.g., Grignard reagent) followed by a suitable reducing agent. In some embodiments, intermediate 5-4 is prepared by a process comprising contacting intermediate 5-2 with compounds of Formula 5-3. In some embodiments, intermediate 5-5 is prepared by a process comprising reacting intermediate 5-4 under suitable conditions. In some embodiments, compounds of Formula 5-6 are prepared by a process comprising reacting intermediate 5-5 under suitable conditions (e.g., nucleophilic aromatic substitution, nucleophilic addition, amide coupling, or transition metal-catalyzed cross-coupling reaction conditions).

In some embodiments, provided compounds are prepared according to the following Scheme:

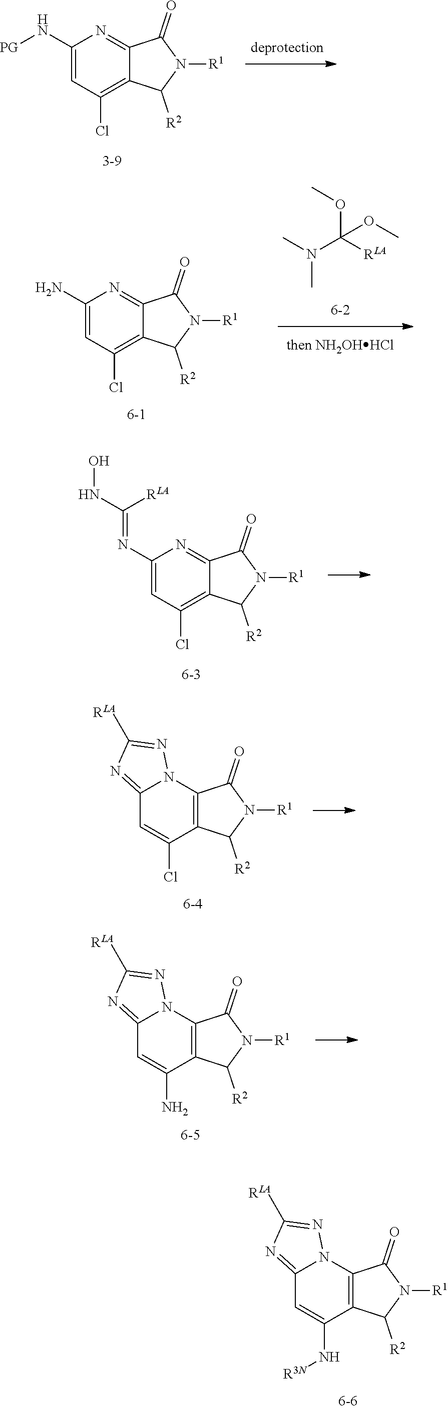

wherein PG is a suitable protecting group (e.g., 2,4-dimethocybenzyl), R^{LA} is -L^{A}-R^{A}, R^{3N} is R^{3A1}, COR^{3A1} or CON(R^{3A1})₂, and each of L^{A}, R^{A}, R¹, R², and R^{3A1} is as defined above for Formula I, and described in classes and subclasses herein, both singly and in combination. Accordingly, in some embodiments, intermediate 6-1 is prepared by a process comprising reacting compounds of Formula 3-9 under suitable conditions (e.g., deprotection conditions). In some embodiments, intermediate 6-3 is prepared by a process comprising contacting intermediate 6-1 with compounds of Formula 6-2 followed by hydroxylamine. In some embodiments, intermediate 6-4 is prepared by a process comprising reacting intermediate 6-3 under suitable conditions. In some embodiments, intermediate 6-5 is prepared by a process comprising reacting intermediate 6-4 under suitable conditions. In some embodiments, compounds of Formula 6-6 are prepared by a process comprising reacting intermediate 6-5 under suitable conditions (e.g. nucleophilic aromatic substitution, nucleophilic addition, amide coupling, or transition metal-catalyzed cross-coupling reaction conditions).

### Compositions

The present disclosure also provides compositions comprising a compound provided herein with one or more other components. In some embodiments, provided compositions comprise and/or deliver a compound described herein (e.g., compounds of any of Formulae I, II, III, and IV).

In some embodiments, a provided composition is a pharmaceutical composition that comprises and/or delivers a compound provided herein (e.g., compounds of any of Formulae I, II, III, and IV) and further comprises a pharmaceutically acceptable carrier.

Pharmaceutical compositions typically contain an active agent (e.g., a compound described herein) in an amount effective to achieve a desired therapeutic effect while avoiding or minimizing adverse side effects. In some embodiments, provided pharmaceutical compositions comprise a compound described herein and one or more fillers, disintegrants, lubricants, glidants, anti-adherents, and/or anti-statics, etc. Provided pharmaceutical compositions can be in a variety of forms including oral dosage forms, topical creams, topical patches, iontophoresis forms, suppository, nasal spray and/or inhaler, eye drops, intraocular injection forms, depot forms, as well as injectable and infusible solutions. Methods of preparing pharmaceutical compositions are well known in the art.

In some embodiments, provided compounds are formulated in a unit dosage form for ease of administration and uniformity of dosage. The expression “unit dosage form” as used herein refers to a physically discrete unit of an active agent (e.g., a compound described herein) for administration to a subject. Typically, each such unit contains a predetermined quantity of active agent. In some embodiments, a unit dosage form contains an entire single dose of the agent. In some embodiments, more than one unit dosage form is administered to achieve a total single dose. In some embodiments, administration of multiple unit dosage forms is required, or expected to be required, in order to achieve an intended effect. A unit dosage form may be, for example, a liquid pharmaceutical composition containing a predetermined quantity of one or more active agents, a solid pharmaceutical composition (e.g., a tablet, a capsule, or the like) containing a predetermined amount of one or more active agents, a sustained release formulation containing a predetermined quantity of one or more active agents, or a drug delivery device containing a predetermined amount of one or more active agents, etc.

Provided compositions may be administered using any amount and any route of administration effective for treating or lessening the severity of any disease or disorder described herein.

### USES

The present disclosure provides uses for compounds and compositions described herein. In some embodiments, provided compounds and compositions are for use in medicine (e.g., as therapy). In some embodiments, provided compounds and compositions are useful in treating a disease, disorder, or condition, wherein an underlying pathology is, wholly or partially, mediated by PI3Kα. In some embodiments, provided compounds and compositions are useful in research as, for example, analytical tools and/or control compounds in biological assays.

In some embodiments, the present disclosure provides methods of administering provided compounds or compositions to a subject in need thereof. In some embodiments, the present disclosure provides methods of administering provided compounds or compositions to a subject suffering from or susceptible to a disease, disorder, or condition associated with PI3Kα. In some embodiments, the present disclosure provides methods of administering provided compounds or compositions to a subject suffering from or susceptible to a disease, disorder, or condition, wherein an underlying pathology is, wholly or partially, mediated by PI3Kα.

In some embodiments, provided compounds are useful as PI3Kα inhibitors. In some embodiments, the present disclosure provides methods of inhibiting PI3Kα in a subject comprising administering a provided compound or composition. In some embodiments, the present disclosure provides methods of inhibiting PI3Kα in a biological sample comprising contacting the sample with a provided compound or composition.

In some embodiments, the present disclosure provides methods of treating a disease, disorder or condition associated with PI3Kα in a subject in need thereof, comprising administering to the subject a provided compound or composition. In some embodiments, a disease, disorder or condition is associated with mutation of PI3Kα. In some embodiments, the present disclosure provides methods of treating a disease, disorder or condition, wherein an underlying pathology is, wholly or partially, mediated by PI3Kα, in a subject in need thereof, comprising administering to the subject a provided compound or composition.

In some embodiments, the present disclosure provides methods of treating a variety of PI3Kα-dependent diseases and disorders. In some embodiments, the disease of disorder is a cancer (e.g., breast cancer, brain cancer, prostate cancer, endometrial cancer, gastric cancer, leukemia, lymphoma, sarcoma, colorectal cancer, lung cancer, ovarian cancer, skin cancer, and head and neck cancer). In some embodiments, the disease or disorder associated with PI3Kα includes, but is not limited to, CLOVES syndrome (congenital lipomatous overgrowth, vascular malformations, epidermal naevi, scoliosis/skeletal and spinal syndrome), PIK3CA-related overgrowth syndrome (PROS), endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

### Preparation of Provided Compounds

### Example 1: N-(6-(2-chloro-5-fluorophenyl)-8-oxo-7,8-dihydro-6H-pyrrolo[3,4-e][1,2,4]triazolo[4,3-a]pyridin-5-yl)-3-fluoro-5-(trifluoromethyl)benzamide (I-1)

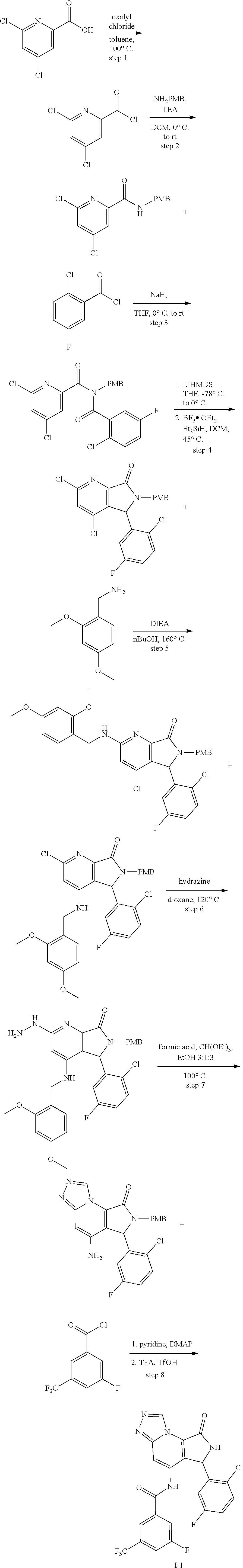

### Step 1: 4,6-dichloropicolinoyl chloride

To a mixture of 4,6-dichloropicolinic acid (14.8 g, 77.08 mmol) in toluene (500 mL) were added oxalyl chloride (19.57 g, 154.2 mmol) and 0.5 mL DMF. The resulting mixture was stirred at 100° C. for 3 h. Upon cooling to room temperature, the reaction was concentrated under reduced pressure. The residue was used directly in next step without further purification.

### Step 2: 4,6-dichloro-N-(4-methoxybenzyl)picolinamide

The mixture of 4,6-dichloropicolinoyl chloride (14.5 g, 70 mmol) in DCM (500 mL) was added TEA (23.4 g, 231.2 mmol) and (4-methoxyphenyl)methanamine (21.15 g, 154.2 mmol) at 0° C. The reaction was then slowly warmed to rt and stirred for 12 h. After completion, the reaction was quenched by added water. The mixture was extracted with dichloromethane (2×300 mL). The combined organics were dried with anhydrous sodium sulfate, filtered, and concentrated under reduced pressure. The residue was purified by silica gel column chromatography, eluted with hexanes and ethyl acetate to provide the desired product as a white solid (18.6 g, 85%). LCMS calculated for C₁₄H₁₃Cl₂N₂O₂ (M+H)⁺ m/z=311.0; found 311.1.

### Step 3: 4,6-dichloro-N-(2-chloro-5-fluorobenzoyl)-N-(4-methoxybenzyl)picolinamide

To a mixture of 4,6-dichloro-N-(4-methoxybenzyl)picolinamide (19.8 g, 63.63 mmol) in THE (500 mL) was added NaH (3.8 g, 95.4 mmol) at 0° C. under nitrogen atmosphere. The resulting mixture was stirred at same temperature for 1 h before 2-chloro-5-fluorobenzoyl chloride (18.42 g, 95.45 mmol) was added. The reaction mixture was then slowly warmed to rt and stirred overnight. After completion, the reaction was carefully quenched with water at 0° C. while vigorously stirred. The mixture was then extracted with ethyl acetate (2×300 mL). The combined organics were dried with anhydrous sodium sulfate, filtered, and concentrated under reduced pressure. The residue was purified by silica gel column chromatography, eluted with hexanes and ethyl acetate to provide the desired product, which was further purified by recrystallization in hexanes and ethyl acetate to provide the pure product as a white solid (25.6 g, 86%). LCMS calculated for C₂₁H₁₅Cl₃FN₂O₃(M+H)⁺ m/z=467.0; found 467.0.

### Step 4: 2,4-dichloro-5-(2-chloro-5-fluorophenyl)-6-(4-methoxybenzyl)-5,6-dihydro-7H-pyrrolo[3,4-b]pyridin-7-one

4,6-dichloro-N-(2-chloro-5-fluorobenzoyl)-N-(4-methoxybenzyl)picolinamide (12.5 g, 26.73 mmol) was dissolved in 300 mL dry THF. The mixture was cooled to −78° C. before LiHMDS (1M, 34.74 mL, THF solution) was added. The reaction was warmed slowly to 0° C., stirred for 10 min then quenched with sat. NH₄Cl solution. The mixture was then extracted with ethyl acetate (2×300 mL). The combined organics were dried with anhydrous sodium sulfate, filtered, and concentrated under reduced pressure. The residue was added 200 mL DCM. The crude product precipitated out and was collected by filtration followed by washing with DCM (2×10 mL) and dried at room temperature to give a brown solid, which was then dissolved in DCM (300 mL). To the above solution, boron trifluoride diethyl etherate (6.6 mL, 53.45 mmol) was added, followed by triethylsilane (20.4 mL, 133.6 mmol). The mixture was then heated to 45° C. and stirred overnight. Upon cooling to room temperature, the reaction was quenched with sat. NaHCO₃, then extracted with DCM (2×300 mL). The combined organics were dried with anhydrous sodium sulfate, filtered, and concentrated under reduced pressure. The residue was purified by silica gel column chromatography, eluted with hexanes and ethyl acetate to provide the desired product as a brown solid (5.8 g, 48%). LCMS calculated for C₂₁H₁₅Cl₃FN₂O₂(M+H)⁺ m/z=451.0; found 451.0.

### Step 5: 2-chloro-5-(2-chloro-5-fluorophenyl)-4-((2,4-dimethoxybenzyl)amino)-6-(4-methoxybenzyl)-5,6-dihydro-7H-pyrrolo[3,4-b]pyridin-7-one

To a mixture of 2,4-dichloro-5-(2-chloro-5-fluorophenyl)-6-(4-methoxybenzyl)-5,6-dihydro-7H-pyrrolo[3,4-b]pyridin-7-one (2.4 g, 5.3 mmol) in n-BuOH (50 mL) was added (2,4-dimethoxyphenyl)methanamine (1.3 g, 7.97 mmol) and N,N-diisopropylethylamine (1.96 mL, 10.6 mmol). The resulting mixture was stirred at 160° C. for 6 h. Upon cooling to room temperature, the mixture was concentrated under reduced pressure. The residue was purified by silica gel column chromatography, eluted with hexanes and ethyl acetate to provide the desired product as a brown oil (1.3 g, 43%). LCMS calculated for C₃₀H₂₇Cl₂FN₃O₄ (M+H)⁺ m/z=582.1; found 582.1. Another isomer was isolated as a brown oil (0.6 g, 21%).

### Step 6: 5-(2-chloro-5-fluorophenyl)-4-((2,4-dimethoxybenzyl)amino)-2-hydrazineyl-6-(4-methoxybenzyl)-5,6-dihydro-7H-pyrrolo[3,4-b]pyridin-7-one

To a mixture of 2-chloro-5-(2-chloro-5-fluorophenyl)-4-((2,4-dimethoxybenzyl)amino)-6-(4-methoxybenzyl)-5,6-dihydro-7H-pyrrolo[3,4-b]pyridin-7-one (200 mg, 0.34 mmol) in dioxane (3 mL) was added hydrazine monohydrate (0.5 mL). The resulting mixture was stirred at 120° C. for 16 h. Upon cooling to room temperature, the mixture was concentrated under reduced pressure. The residue was purified by silica gel column chromatography, eluted with DCM and MeOH to provide the desired product as a white solid (133 mg, 68%). LCMS calculated for C₃₀H₃₀ClFN₅O₄(M+H)⁺ m/z=578.2; found 578.3.

### Step 7: 5-amino-6-(2-chloro-5-fluorophenyl)-7-(4-methoxybenzyl)-6,7-dihydro-8H-pyrrolo[3,4-e][1,2,4]triazolo[4,3-a]pyridin-8-one

5-(2-chloro-5-fluorophenyl)-4-((2,4-dimethoxybenzyl)amino)-2-hydrazineyl-6-(4-methoxybenzyl)-5,6-dihydro-7H-pyrrolo[3,4-b]pyridin-7-one (50 mg, 0.086 mmol) in a 2 dram vial was added formic acid (1 mL), triethyl orthoformate (0.3 mL), and EtOH (1 mL). The mixture was heated to 100° C. for 12 h. Upon cooling to room temperature, the mixture was concentrated under reduced pressure. The residue was purified by silica gel column chromatography, eluted with DCM and MeOH to provide the desired product as a white solid (18 mg, 50%). LCMS calculated for C₂₂H₁₈ClFN₅O₂(M+H)⁺ m/z=438.1; found 438.2.

### Step 8: N-(6-(2-chloro-5-fluorophenyl)-8-oxo-7,8-dihydro-6H-pyrrolo[3,4-e][1,2,4]triazolo[4,3-a]pyridin-5-yl)-3-fluoro-5-(trifluoromethyl)benzamide

In a 2 dram vial equipped with stir bar was added 5-amino-6-(2-chloro-5-fluorophenyl)-7-(4-methoxybenzyl)-6,7-dihydro-8H-pyrrolo[3,4-e][1,2,4]triazolo[4,3-a]pyridin-8-one (9 mg, 0.02 mmol), pyridine (0.5 mL), DMAP (2 mg, 0.016 mmol). The mixture was cooled to 0° C. and added 3-fluoro-5-(trifluoromethyl)benzoyl chloride (7 mg, 0.03 mmol). The reaction was warmed to rt and stirred for 1 h, then quenched with water. The mixture was then extracted with EtOAc, and the combined organics were dried with anhydrous sodium sulfate, filtered, and concentrated under reduced pressure. The residue was then dissolved in 20% TfOH in TFA, and heated to 70° C. for 10 min. The reaction was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₂H₁₂ClF₅N₅O₂ (M+H)⁺ m/z=508.1; found 508.1.

### Example 2: N-(3-(2-chloro-5-fluorophenyl)-1-oxo-2,3-dihydro-1H-imidazo[1,2-a]pyrrolo[3,4-e]pyridin-4-yl)-3-fluoro-5-(trifluoromethyl)benzamide (I-2)

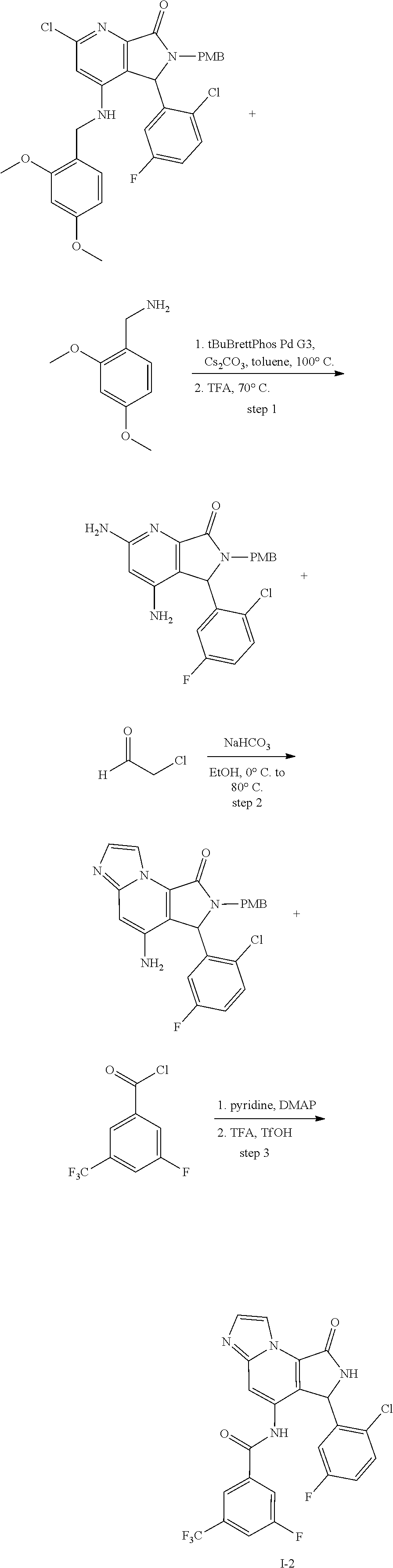

### Step 1: 2,4-diamino-5-(2-chloro-5-fluorophenyl)-6-(4-methoxybenzyl)-5,6-dihydro-7H-pyrrolo[3,4-b]pyridin-7-one

The mixture of 2-chloro-5-(2-chloro-5-fluorophenyl)-4-((2,4-dimethoxybenzyl)amino)-6-(4-methoxybenzyl)-5,6-dihydro-7H-pyrrolo[3,4-b]pyridin-7-one (350 mg, 0.6 mmol), tBuBrettPhos Pd G3 (54 mg, 0.06 mmol), (2,4-dimethoxyphenyl)methanamine (150 mg, 0.9 mmol) and Cs₂CO₃ (293 mg, 0.9 mmol) in toluene (5 mL) was heated at 100° C. for 12 h under nitrogen atmosphere. Upon cooling to room temperature, the mixture was filtered, and concentrated under reduced pressure. The residue was purified by silica gel column chromatography, eluted with DCM and ethyl acetate (0 to 90%), and concentrated. The residue was then dissolved in TFA and heated to 70° C. for 20 min. The resulting mixture was washed with sat. NaHCO₃ and extracted with DCM. The combined organics were dried with anhydrous sodium sulfate, filtered, and concentrated under reduced pressure to provide the crude product as a white solid (74 mg). LCMS calculated for C₂₁H₁₉ClFN₄O₂(M+H)⁺ m/z=413.1; found 413.2.

### Step 2: 4-amino-3-(2-chloro-5-fluorophenyl)-2-(4-methoxybenzyl)-2,3-dihydro-1H-imidazo[1,2-a]pyrrolo[3,4-e]pyridin-1-one

In a 2 dram vial equipped with stir bar was added 2,4-diamino-5-(2-chloro-5-fluorophenyl)-6-(4-methoxybenzyl)-5,6-dihydro-7H-pyrrolo[3,4-b]pyridin-7-one (70 mg, 0.15 mmol), NaHCO₃ (26 mg, 0.31 mmol) and EtOH (1.5 mL). 2-Chloroacetaldehyde water solution (50%, 70 mg) was then added at 0° C. The reaction mixture was then warmed to 80° C. and stirred 2 h. After completion, the reaction was quenched with water and extracted with DCM. The combined organics were dried with anhydrous sodium sulfate, filtered, and concentrated under reduced pressure to provide the crude product as a white solid (26 mg). LCMS calculated for C₂₃H₁₉ClFN₄O₂(M+H)⁺ m/z=437.1; found 437.2.

### Step 3: N-(3-(2-chloro-5-fluorophenyl)-1-oxo-2,3-dihydro-1H-imidazo[1,2-a]pyrrolo[3,4-e]pyridin-4-yl)-3-fluoro-5-(trifluoromethyl)benzamide

In a 2 dram vial equipped with stir bar was added 4-amino-3-(2-chloro-5-fluorophenyl)-2-(4-methoxybenzyl)-2,3-dihydro-1H-imidazo[1,2-a]pyrrolo[3,4-e]pyridin-1-one (9 mg, 0.02 mmol), pyridine (0.5 mL), DMAP (2 mg, 0.016 mmol). The mixture was cooled to 0° C. and added 3-fluoro-5-(trifluoromethyl)benzoyl chloride (7 mg, 0.03 mmol). The reaction was warmed to rt and stirred for 1 h, then quenched with water. The mixture was then extracted with EtOAc, the combined organics were dried with anhydrous sodium sulfate, filtered, and concentrated under reduced pressure. The residue was then dissolved in 20% TfOH in TFA, and heated to 70° C. for 10 min. The reaction was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₁₃ClF₅N₄O₂ (M+H)⁺ m/z=507.1; found 507.1.

### Example 3: N-(6-(2-chloro-5-fluorophenyl)-8-oxo-7,8-dihydro-6H-imidazo[1,5-a]pyrrolo[3,4-e]pyridin-5-yl)-3-fluoro-5-(trifluoromethyl)benzamide (I-3)

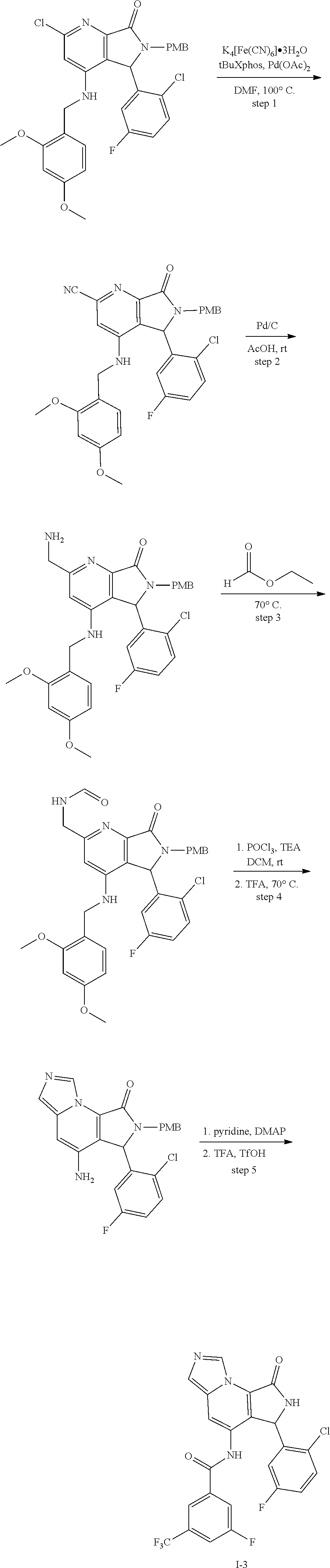

### Step 1: 5-(2-chloro-5-fluorophenyl)-4-((2,4-dimethoxybenzyl)amino)-6-(4-methoxybenzyl)-7-oxo-6,7-dihydro-5H-pyrrolo[3,4-b]pyridine-2-carbonitrile

The mixture of 2-chloro-5-(2-chloro-5-fluorophenyl)-4-((2,4-dimethoxybenzyl)amino)-6-(4-methoxybenzyl)-5,6-dihydro-7H-pyrrolo[3,4-b]pyridin-7-one (160 mg, 0.27 mmol), tBuXPhos (25 mg, 0.06 mmol), Pd(OAc)₂ (13 mg, 0.05 mmol) and potassium hexacyanoferrate(II) trihydrate (232 mg, 0.55 mmol) in DMF (3 mL) was heated at 100° C. for 12 h under nitrogen atmosphere. Upon cooling to room temperature, the mixture was filtered, and concentrated under reduced pressure. The residue was purified by silica gel column chromatography, eluted with hexanes and ethyl acetate (0 to 50%) to provide the desired product as a white solid (74 mg, 50%). LCMS calculated for C₃₁H₂₇ClFN₄O₄ (M+H)⁺ m/z=573.2; found 573.2.

### Step 2: 2-(aminomethyl)-5-(2-chloro-5-fluorophenyl)-4-((2,4-dimethoxybenzyl)amino)-6-(4-methoxybenzyl)-5,6-dihydro-7H-pyrrolo[3,4-b]pyridin-7-one

To a mixture of 5-(2-chloro-5-fluorophenyl)-4-((2,4-dimethoxybenzyl)amino)-6-(4-methoxybenzyl)-7-oxo-6,7-dihydro-5H-pyrrolo[3,4-b]pyridine-2-carbonitrile (74 mg, 0.13 mmol), Pd/C (15 mg, 0.01 mmol) in AcOH (2 mL) was charged with 1 atm H₂. The reaction was stirred at rt for 6 h. Upon completion, the mixture was filtered, and concentrated under reduced pressure. The residue was diluted with DCM and washed with sat. NaHCO₃, the mixture was then extracted with DCM. The combined organics were dried with anhydrous sodium sulfate, filtered, and concentrated under reduced pressure. The residue was purified by silica gel column chromatography, eluted with DCM and MeOH (0 to 10%) to provide the desired product as a light yellow solid (60 mg, 80%). LCMS calculated for C₃₁H₃₁ClFN₄O₄ (M+H)⁺ m/z=577.2; found 577.2.

### Step 3: N-((5-(2-chloro-5-fluorophenyl)-4-((2,4-dimethoxybenzyl)amino)-6-(4-methoxybenzyl)-7-oxo-6,7-dihydro-5H-pyrrolo[3,4-b]pyridin-2-yl)methyl)formamide

In a 2 dram vial equipped with stir bar was added 2-(aminomethyl)-5-(2-chloro-5-fluorophenyl)-4-((2,4-dimethoxybenzyl)amino)-6-(4-methoxybenzyl)-5,6-dihydro-7H-pyrrolo[3,4-b]pyridin-7-one (60 mg, 0.1 mmol), and ethyl formate (2 mL). The mixture was heated to 65° C. for 16 h. Upon cooling to room temperature, the mixture was concentrated under reduced pressure to provide the crude product as a white solid which was used directly in next step. LCMS calculated for C₃₂H₃₁ClFN₄O₅(M+H)⁺ m/z=605.2; found 605.2.

### Step 4: 5-amino-6-(2-chloro-5-fluorophenyl)-7-(4-methoxybenzyl)-6,7-dihydro-8H-imidazo[1,5-a]pyrrolo[3,4-e]pyridin-8-one

In a 2 dram vial equipped with stir bar was added N-((5-(2-chloro-5-fluorophenyl)-4-((2,4-dimethoxybenzyl)amino)-6-(4-methoxybenzyl)-7-oxo-6,7-dihydro-5H-pyrrolo[3,4-b]pyridin-2-yl)methyl)formamide (50 mg, 0.09 mmol), and DCM (2 mL). The mixture was cooled to 0° C. and added POCl₃ (17.3 mg, 0.11 mmol) and TEA (43.8 mg, 0.42 mmol) sequentially. The reaction was warmed to rt then stirred for 1 h before quenched with sat. NaHCO₃. The mixture was then extracted with DCM. The combined organics were dried with anhydrous sodium sulfate, filtered, and concentrated under reduced pressure. The residue was dissolved in TFA and heated to 70° C. for 10 min. The reaction was warmed to rt then stirred for 1 h before quenched with sat. NaHCO₃. The mixture was then extracted with DCM. The combined organics were dried with anhydrous sodium sulfate, filtered, and concentrated under reduced pressure. The residue was purified by silica gel column chromatography, eluted with DCM and MeOH (0 to 10%) to provide the desired product as a light yellow solid (19.6 mg, 50%). LCMS calculated for C₂₃H₁₉ClFN₄O₂(M+H)⁺ m/z=437.2; found 437.2.

### Step 5: N-(6-(2-chloro-5-fluorophenyl)-8-oxo-7,8-dihydro-6H-imidazo[1,5-a]pyrrolo[3,4-e]pyridin-5-yl)-3-fluoro-5-(trifluoromethyl)benzamide

In a 2 dram vial equipped with stir bar was added 5-amino-6-(2-chloro-5-fluorophenyl)-7-(4-methoxybenzyl)-6,7-dihydro-8H-imidazo[1,5-a]pyrrolo[3,4-e]pyridin-8-one (12 mg, 0.03 mmol), pyridine (0.5 mL), DMAP (2 mg, 0.016 mmol). The mixture was cooled to 0° C. and added 3-fluoro-5-(trifluoromethyl)benzoyl chloride (8 mg, 0.03 mmol). The reaction was warmed to rt and stirred for 1 h, then quenched with water. The mixture was then extracted with EtOAc, and the combined organics were dried with anhydrous sodium sulfate, filtered, and concentrated under reduced pressure. The residue was then dissolved in 20% TfOH in TFA, and heated to 70° C. for 10 min. The reaction was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₃H₁₃ClF₅N₄O₂ (M+H)⁺ m/z=507.1; found 507.1.

### Example 4: N-(6-(2-chloro-5-fluorophenyl)-8-oxo-7,8-dihydro-6H-pyrrolo[3,4-e][1,2,4]triazolo[1,5-a]pyridin-5-yl)-3-fluoro-5-(trifluoromethyl)benzamide (I-4)

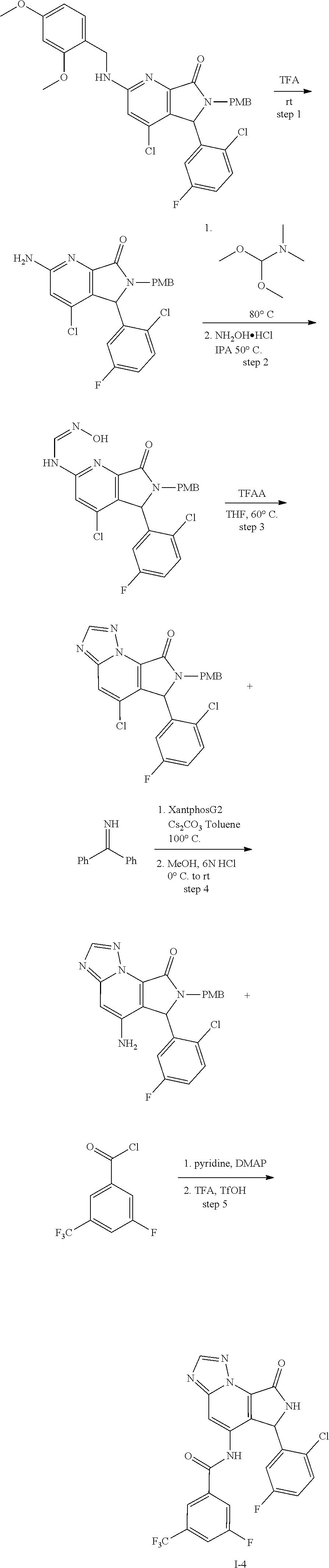

### Step 1: 2-amino-4-chloro-5-(2-chloro-5-fluorophenyl)-6-(4-methoxybenzyl)-5,6-dihydro-7H-pyrrolo[3,4-b]pyridin-7-one

In a 2 dram vial equipped with stir bar was added 4-chloro-5-(2-chloro-5-fluorophenyl)-2-((2,4-dimethoxybenzyl)amino)-6-(4-methoxybenzyl)-5,6-dihydro-7H-pyrrolo[3,4-b]pyridin-7-one (Example 1, step 5) (600 mg, 1.03 mmol), TFA (2 mL). The mixture was stirred at rt for 3 h. The mixture was concentrated under reduced pressure and added sat. NaHCO₃. The resulting mixture is filtrated, and the white solid was collected as desired product which was used directly in next step. LCMS calculated for C₂₁H₁₇Cl₂FN₃O₂ (M+H)⁺ m/z=432.1; found 432.1.

### Step 2: N-(4-chloro-5-(2-chloro-5-fluorophenyl)-6-(4-methoxybenzyl)-7-oxo-6,7-dihydro-5H-pyrrolo[3,4-b]pyridin-2-yl)-N′-hydroxyformimidamide

In a flask containing a magnetic stir bar, 2-amino-4-chloro-5-(2-chloro-5-fluorophenyl)-6-(4-methoxybenzyl)-5,6-dihydro-7H-pyrrolo[3,4-b]pyridin-7-one (400 mg, 0.92 mmol) was dissolved in 2-propanol (5 mL). To this solution was added 1,1-dimethoxy-N,N-dimethylmethanamine (0.31 mL, 2.31 mmol). The reaction mixture was stirred at 80° C. for 2 h. The mixture was cooled to room temperature and hydroxylamine hydrochloride (160 mg, 2.31 mmol) was added. The reaction mixture was stirred at 50° C. After 2 h, the mixture was concentrated under reduced pressure and the resulting solid was used in the next reaction without further purification. LCMS calculated for C₂₂H₁₈Cl₂FN₄O₃(M+H)⁺ m/z=475.1; found 475.1.

### Step 3: 5-chloro-6-(2-chloro-5-fluorophenyl)-7-(4-methoxybenzyl)-6,7-dihydro-8H-pyrrolo[3,4-e][1,2,4]triazolo[1,5-a]pyridin-8-one

In a flask containing a magnetic stir bar, N-(4-chloro-5-(2-chloro-5-fluorophenyl)-6-(4-methoxybenzyl)-7-oxo-6,7-dihydro-5H-pyrrolo[3,4-b]pyridin-2-yl)-N′-hydroxyformimidamide (300 mg, 0.63 mmol) was dissolved in THE (3 mL). The solution was cooled to 0° C., and trifluoroacetic anhydride (1.62 mL, 11.5 mmol) was added. The reaction mixture was stirred at 60° C. for 12 h. Saturated aqueous NaHCO₃ was added. The mixture was extracted with DCM. The combined organic layers were concentrated in vacuo. The residue was purified by chromatography to afford the title compound as a white solid (120 mg 41%). LCMS calculated for C₂₂H₁₆Cl₂FN₄O₂(M+H)⁺ m/z=457.1; found 457.1.

### Step 4: 5-amino-6-(2-chloro-5-fluorophenyl)-7-(4-methoxybenzyl)-6,7-dihydro-8H-pyrrolo[3,4-e][1,2,4]triazolo[1,5-a]pyridin-8-one

The mixture of 5-chloro-6-(2-chloro-5-fluorophenyl)-7-(4-methoxybenzyl)-6,7-dihydro-8H-pyrrolo[3,4-e][1,2,4]triazolo[1,5-a]pyridin-8-one (110 mg, 0.24 mmol), Xantphos Pd G2 (17 mg, 0.02 mmol), diphenylmethanimine (84 mg, 0.48 mmol) and Cs₂CO₃ (117 mg, 0.36 mmol) in toluene (2 mL) was heated at 100° C. for 6 h under nitrogen atmosphere. Upon cooling to room temperature, the mixture was filtered, and concentrated under reduced pressure. The residue was purified by silica gel column chromatography, eluted with DCM and EtOAc (0 to 90%) and concentrated. The residue was then dissolved in MeOH and added 6 N HCl at 0° C. The reaction mixture was stirred for 20 min, which was then washed with sat. NaHCO₃ and extracted with DCM/IPA. The combined organics were dried with anhydrous sodium sulfate, filtered, and concentrated under reduced pressure. The residue was purified by silica gel column chromatography, eluted with DCM and MeOH (0 to 10%) to provide the desired product as a white solid (40 mg, 38%). LCMS calculated for C₂₂H₁₈ClFN₅O₂(M+H)+m/z=438.1; found 438.2.

### Step 5: N-(6-(2-chloro-5-fluorophenyl)-8-oxo-7,8-dihydro-6H-pyrrolo[3,4-e][1,2,4]triazolo[1,5-a]pyridin-5-yl)-3-fluoro-5-(trifluoromethyl)benzamide

In a 2 dram vial equipped with stir bar was added 5-amino-6-(2-chloro-5-fluorophenyl)-7-(4-methoxybenzyl)-6,7-dihydro-8H-pyrrolo[3,4-e][1,2,4]triazolo[1,5-a]pyridin-8-one (12 mg, 0.03 mmol), pyridine (0.5 mL), DMAP (2 mg, 0.016 mmol). The mixture was cooled to 0° C. and added 3-fluoro-5-(trifluoromethyl)benzoyl chloride (8 mg, 0.03 mmol). The reaction was warmed to rt and stirred for 1 h, then quenched with water. The mixture was then extracted with EtOAc, and the combined organics were dried with anhydrous sodium sulfate, filtered, and concentrated under reduced pressure. The residue was then dissolved in 20% TfOH in TFA, and heated to 70° C. for 10 min. The reaction was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the desired product as a white solid. LCMS calculated for C22H₁₂ClF₅N₅O₂ (M+H)⁺ m/z=508.1; found 508.1.

### Example 5: N-(7-(2-chloro-5-fluorophenyl)-9-oxo-8,9-dihydro-7H-imidazo[1,2-a]pyrrolo[3,4-c]pyridin-6-yl)-3-fluoro-5-(trifluoromethyl)benzamide (I-5)

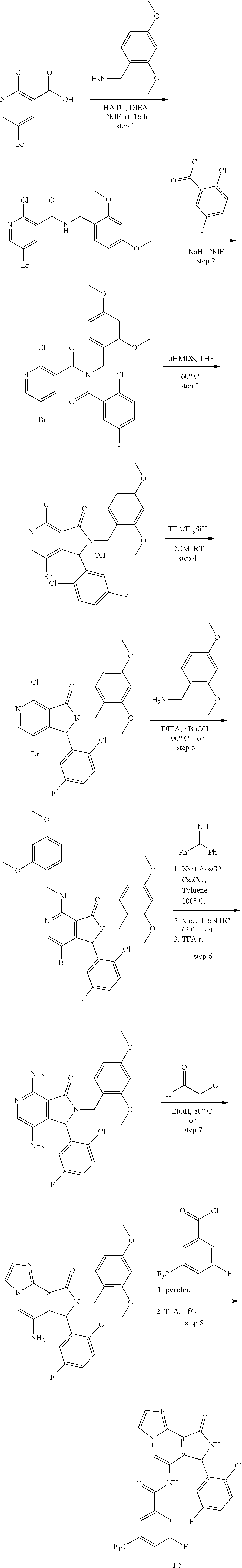

### Step 1: 5-Bromo-2-chloro-N-(2,4-dimethoxybenzyl)nicotinamide

The mixture of 5-bromo-2-chloronicotinic acid (30 g, 126.88 mmol) in N,N-dimethylformamide (300 mL) was treated with N,N,N,N-tetramethyl-O-(7-azabenzotriazol-1-yl)uronium hexafluorophospate (57.89 g, 152.25 mmol) at room temperature for 20 min, followed by the addition of 1-(2,4-dimethoxyphenyl)methanamine (22.28 g, 133.22 mmol) and N-ethyl-N-isopropylpropan-2-amine (49.2 g, 380.63 mmol). The resulting mixture was stirred at the same temperature for additional 16 h, and then diluted with ethyl acetate (3 L). The resulting mixture was washed with water (3×3 L) and brine (3×1 L). The combined organics were dried with anhydrous sodium sulfate. After filtration, the filtrate was concentrated under reduced pressure. The residue was purified by silica gel column chromatography, eluted with 80% dichloromethane in petroleum ether to provide the desired product as a white solid (33 g, 67%). LCMS calculated for C₁₅H₁₅BrClN₂O₃(M+H)⁺ m/z=385.0; found 384.9; ¹H NMR (400 MHz, CDCl₃) δ 8.48 (d, J=2.8 Hz, 1H), 8.25 (d, J=2.8 Hz, 1H), 7.26-7.24 (m, 1H), 7.04 (s, 1H), 6.48-6.45 (m, 2H), 4.58 (d, J=5.6 Hz, 2H), 3.85 (s, 3H), 3.81 (s, 3H).

### Step 2: 5-Bromo-2-chloro-N-(2-chloro-5-fluorobenzoyl)-N-(2,4-dimethoxybenzyl)nicotinamide

The mixture of 2-chloro-5-fluorobenzoic acid (3.85 g, 22.04 mmol) in anhydrous tetrahydrofuran (40 mL) was treated with oxalyl chloride (4.11 g, 32.41 mmol) and N,N-dimethylformamide (2 mL) dropwise at 0° C. under nitrogen atmosphere. The resulting mixture was stirred at room temperature for 1 h, and then concentrated under reduced pressure to give acid chloride. The mixture of 5-bromo-2-chloro-N-(2,4-dimethoxybenzyl)nicotinamide (5 g, 12.97 mmol) in N,N-dimethylformamide (50 mL) was treated with sodium hydride (60% in mineral oil, 1.04 g, 25.93 mmol) in portions at 0° C. under nitrogen atmosphere. After stirring for 30 min at the same temperature, acid chloride (prepared above) was added dropwise. The resulting mixture was stirred at room temperature for additional 2 h, and then quenched by the addition of saturated aqueous ammonium chloride (100 mL). The resulting mixture was extracted with ethyl acetate (3×100 mL). The combined organic layers were dried with anhydrous sodium sulfate. After filtration, the filtrate was concentrated under reduced pressure. The residue was purified by silica gel column chromatography, eluted with 40% ethyl acetate in petroleum ether to provide the desired product as a yellow solid (5.5 g, 78%). LCMS calculated for C₂₂H₁₇BrCl₂FN₂O₄(M+H)⁺ m/z=541.0; found 540.9; ¹H NMR (400 MHz, CDCl₃) δ 8.36 (d, J=2.4 Hz, 1H), 7.60 (d, J=2.4 Hz, 1H), 7.26-7.23 (m, 2H), 7.04-6.95 (m, 2H), 6.49-6.46 (m, 1H), 6.41-6.39 (m, 1H), 5.05 (s, 2H), 3.81 (s, 3H), 3.69 (s, 3H).

### Step 3: 7-Bromo-4-chloro-1-(2-chloro-5-fluorophenyl)-2-(2,4-dimethoxybenzyl)-1-hydroxy-1,2-dihydro-3H-pyrrolo[3,4-c]pyridin-3-one

The mixture of 5-bromo-2-chloro-N-(2-chloro-5-fluorobenzoyl)-N-(2,4-dimethoxybenzyl)nicotinamide (3.18 g, 5.865 mmol, 1 equiv) in anhydrous tetrahydrofuran (40 mL) was treated with lithium bis(trimethylsilyl)amide (1 M in tetrahydrofuran, 8.8 mL, 8.8 mmol) dropwise at −60° C. under nitrogen atmosphere. After stirring at the same temperature for 1 h, the reaction was quenched by the addition of saturated aqueous ammonium chloride (50 mL). The resulting mixture was extracted with ethyl acetate (3×100 mL). The combined organic layers were dried with anhydrous sodium sulfate. After filtration, the filtrate was concentrated under reduced pressure. The residue was purified by silica gel column chromatography, eluted with 40% ethyl acetate in petroleum ether to provide the desired product as a light yellow solid (2.7 g, 85%). LCMS calculated for C₂₂H₁₇BrCl₂FN₂O₄(M+H)⁺ m/z=541.0; found 540.9; ¹H NMR (300 MHz, CDCl₃) δ 8.54 (s, 1H), 8.07 (dd, J=9.9, 3.0 Hz, 1H), 7.45 (d, J=8.4 Hz, 1H), 7.23-7.18 (m, 1H), 7.11-7.05 (m, 1H), 6.49-6.46 (m, 1H), 6.39-6.38 (m, 1H), 5.13 (s, 1H), 4.62-4.57 (m, 1H), 4.11-4.06 (m, 1H), 3.87 (s, 3H), 3.79 (s, 3H).

### Step 4: 7-Bromo-4-chloro-1-(2-chloro-5-fluorophenyl)-2-(2,4-dimethoxybenzyl)-1,2-dihydro-3H-pyrrolo[3,4-c]pyridin-3-one

The mixture of 7-bromo-4-chloro-1-(2-chloro-5-fluorophenyl)-2-(2,4-dimethoxybenzyl)-1-hydroxy-1,2-dihydro-3H-pyrrolo[3,4-c]pyridin-3-one (3.5 g, 6.46 mmol) in dichloromethane (50 mL) was treated with 2,2,2-trifluoroacetic acid (22.13 g, 194.1 mmol) and triethylsilane (11.26 g, 96.83 mmol) dropwise at 0° C. under nitrogen atmosphere. After stirring at room temperature for 5 days, the mixture was allowed to cool down to 0° C., neutralized to pH=8 with saturated aqueous sodium carbonate. The resulting mixture was extracted with dichloromethane (3×100 mL), the combined organic layers were dried with anhydrous sodium sulfate. After filtration, the filtrate was concentrated under reduced pressure. The residue was purified by reverse flash chromatography (column, C18 silica gel; mobile phase, methanol in water (10 mmol/L ammonium bicarbonate), 5% to 95% gradient over 45 min; detector, UV 254 nm). The fractions were collected, combined and lyophilized to provide the desired product as a light yellow solid (2.54 g, 75%). LCMS calculated for C₂₂H₁₇BrCl₂FN₂O₃(M+H)⁺ m/z=525.0; found 525.0; ¹H NMR (300 MHz, CDCl₃) δ 8.54 (s, 1H), 7.43-7.39 (m, 1H), 7.16-7.14 (m, 1H), 7.06-6.99 (m, 1H), 6.44-6.36 (m, 3H), 5.96 (s, 1H), 4.93 (d, J=14.7 Hz, 1H), 4.09 (d, J=14.7 Hz, 1H), 3.80 (s, 3H), 3.73 (s, 3H).

### Step 5: 7-bromo-1-(2-chloro-5-fluorophenyl)-2-(2,4-dimethoxybenzyl)-4-((2,4-dimethoxybenzyl)amino)-1,2-dihydro-3H-pyrrolo[3,4-c]pyridin-3-one

To a mixture of 7-bromo-4-chloro-1-(2-chloro-5-fluorophenyl)-2-(2,4-dimethoxybenzyl)-1,2-dihydro-3H-pyrrolo[3,4-c]pyridin-3-one (200 mg, 0.38 mmol) in n-BuOH (5 mL) was added (2,4-dimethoxyphenyl)methanamine (95 mg, 0.57 mmol) and N,N-diisopropylethylamine (0.1 mL, 0.57 mmol). The resulting mixture was stirred at 100° C. for 16 h. Upon cooling to room temperature, the mixture was concentrated under reduced pressure. The residue was purified by silica gel column chromatography, eluted with hexanes and ethyl acetate to provide the desired product as a pale-yellow oil (210 mg, 84%). LCMS calculated for C₃₁H₂₉BrClFN₃O₅(M+H)⁺ m/z=656.1; found 656.1.

### Step 6: 4,7-diamino-1-(2-chloro-5-fluorophenyl)-2-(2,4-dimethoxybenzyl)-1,2-dihydro-3H-pyrrolo[3,4-c]pyridin-3-one

The mixture of 7-bromo-1-(2-chloro-5-fluorophenyl)-2-(2,4-dimethoxybenzyl)-4-((2,4-dimethoxybenzyl)amino)-1,2-dihydro-3H-pyrrolo[3,4-c]pyridin-3-one (50 mg, 0.15 mmol), Xantphos Pd G2 (8 mg, 0.01 mmol), diphenylmethanimine (42 mg, 0.24 mmol) and Cs₂CO₃ (60 mg, 0.18 mmol) in toluene (2 mL) was heated at 100° C. for 12 h under nitrogen atmosphere. Upon cooling to room temperature, the mixture was filtered, and concentrated under reduced pressure. The residue was purified by silica gel column chromatography, eluted with DCM and EtOAc (0 to 90%) and concentrated. The residue was then dissolved in MeOH and added 6 N HCl at 0° C. The reaction mixture was stirred for 20 min, which was then washed with sat. NaHCO₃ and extracted with DCM/IPA. The combined organics were dried with anhydrous sodium sulfate, filtered, and concentrated under reduced pressure. The residue was dissolved in TFA and stirred at rt for 2 h, then quenched with sat. NaHCO₃ and extracted with DCM/TPA. The combined organics were dried with anhydrous sodium sulfate, filtered, and concentrated under reduced pressure. The residue was purified by silica gel column chromatography, eluted with DCM and MeOH (0 to 10%) to provide the desired product as a white solid (20 mg, 30%). LCMS calculated for C₂₂H₂₁ClFN₄O₃(M+H)⁺ m/z=443.1; found 443.1.

### Step 7: 6-amino-7-(2-chloro-5-fluorophenyl)-8-(2,4-dimethoxybenzyl)-7,8-dihydro-9H-imidazo[1,2-a]pyrrolo[3,4-c]pyridin-9-one

In a 2 dram vial equipped with stir bar was added 4,7-diamino-1-(2-chloro-5-fluorophenyl)-2-(2,4-dimethoxybenzyl)-1,2-dihydro-3H-pyrrolo[3,4-c]pyridin-3-one (20 mg, 0.04 mmol) and EtOH (0.5 mL), and 2-chloroacetaldehyde water solution (50%, 20 mg) was then added at 0° C. The reaction mixture was then warmed to 80° C. and stirred for 6 h. After completion, the reaction was quenched with water and extracted with DCM. The combined organics were dried with anhydrous sodium sulfate, filtered, and concentrated under reduced pressure to provide the crude product as a white solid (10 mg). LCMS calculated for C₂₄H₂₁ClFN₄O₃(M+H)⁺ m/z=467.1; found 467.2.

### Step 8: N-(7-(2-chloro-5-fluorophenyl)-9-oxo-8,9-dihydro-7H-imidazo[1,2-a]pyrrolo[3,4-c]pyridin-6-yl)-3-fluoro-5-(trifluoromethyl)benzamide

In a 2 dram vial equipped with stir bar was added 6-amino-7-(2-chloro-5-fluorophenyl)-8-(2,4-dimethoxybenzyl)-7,8-dihydro-9H-imidazo[1,2-a]pyrrolo[3,4-c]pyridin-9-one (10 mg, 0.02 mmol), and pyridine (0.5 mL). The mixture was cooled to 0° C. and added 3-fluoro-5-(trifluoromethyl)benzoyl chloride (8 mg, 0.03 mmol). The reaction was warmed to rt and stirred for 1 h, then quenched with water. The mixture was then extracted with EtOAc, and the combined organics were dried with anhydrous sodium sulfate, filtered, and concentrated under reduced pressure. The residue was then dissolved in 20% TfOH in TFA, and stirred at rt for 10 min. The reaction was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of desired product as a white solid. LCMS calculated for C₂₃H₁₃ClF₅N₄O₂ (M+H)⁺ m/z=507.1; found 507.1.

### Example 6: ethyl 3-(2-chloro-5-fluorophenyl)-4-(3-fluoro-5-(trifluoromethyl)benzamido)-1-oxo-2,3-dihydro-1H-imidazo[1,2-a]pyrrolo[3,4-e]pyridine-7-carboxylate (I-6)

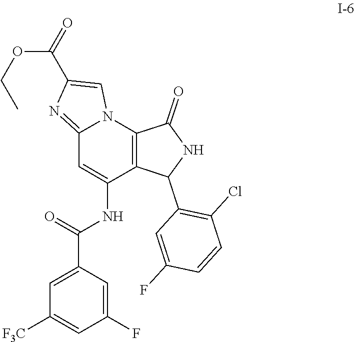 [SMILES: CCOC(=O)c1cn2c3c(c(NC(=O)c4cc(F)cc(C(F)(F)F)c4)cc2n1)C(c1cc(F)ccc1Cl)NC3=O]

The titled compound was prepared using similar procedures as described for Example 2 with ethyl 3-bromo-2-oxopropanoate replacing 2-chloroacetaldehyde in Step 2. The resulting mixture was purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₆H₁₇ClF₅N₄O₄ (M+H)⁺ m/z=579.1; found 579.1.

### Example 7: 3-(2-Chloro-5-fluorophenyl)-4-(3-fluoro-5-(trifluoromethyl)benzamido)-N-methyl-1-oxo-2,3-dihydro-1H-imidazo[1,2-a]pyrrolo[3,4-e]pyridine-7-carboxamide

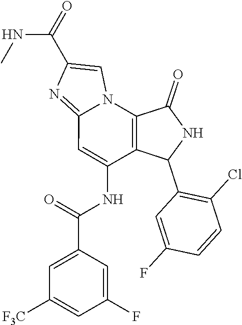 [SMILES: CNC(=O)c1cn2c3c(c(NC(=O)c4cc(F)cc(C(F)(F)F)c4)cc2n1)C(c1cc(F)ccc1Cl)NC3=O]

### Step 1: Ethyl 3-(2-chloro-5-fluorophenyl)-4-(3-fluoro-5-(trifluoromethyl)benzamido)-2-(4-methoxybenzyl)-1-oxo-2,3-dihydro-1H-imidazo[1,2-a]pyrrolo[3,4-e]pyridine-7-carboxylate

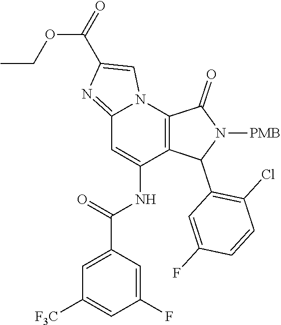

The titled compound was prepared using similar procedures as described for Example 2 with ethyl 3-bromo-2-oxopropanoate replacing 2-chloroacetaldehyde in Step 2. The resulting mixture was purified by silica gel column chromatography to provide the desired product as a white solid. LCMS calculated for C₃₄H₂₅ClF₅N₄O₅ (M+H)⁺ m/z=699.1; found 699.1.

### Step 2: 3-(2-Chloro-5-fluorophenyl)-4-(3-fluoro-5-(trifluoromethyl)benzamido)-2-(4-methoxybenzyl)-1-oxo-2,3-dihydro-1H-imidazo[1,2-a]pyrrolo[3,4-e]pyridine-7-carboxylic acid

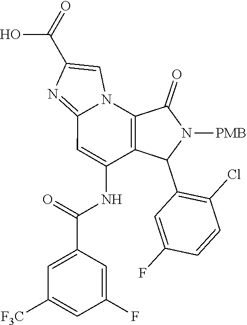

To the mixture of ethyl 3-(2-chloro-5-fluorophenyl)-4-(3-fluoro-5-(trifluoromethyl)benzamido)-2-(4-methoxybenzyl)-1-oxo-2,3-dihydro-1H-imidazo[1,2-a]pyrrolo[3,4-e]pyridine-7-carboxylate (60 mg, 0.08 mmol) in EtOH (2 mL) was added 2N NaOH (1 mL). The resulting mixture was heated to 60° C. for 2 h then cooled to room temperature and acetified by 2N HCl. The mixture was extracted with EtOAc and the combined organics were dried with anhydrous sodium sulfate, filtered, and concentrated under reduced pressure to provide the crude product as a white solid (40 mg) which was used in the next step without further purification. LCMS calculated for C₃₂H₂₁ClF₅N₄O₅ (M+H)⁺ m/z=671.1; found 671.2.

### Step 3: 3-(2-Chloro-5-fluorophenyl)-4-(3-fluoro-5-(trifluoromethyl)benzamido)-N-methyl-1-oxo-2,3-dihydro-1H-imidazo[1,2-a]pyrrolo[3,4-e]pyridine-7-carboxamid

A mixture of 3-(2-chloro-5-fluorophenyl)-4-(3-fluoro-5-(trifluoromethyl)benzamido)-2-(4-methoxybenzyl)-1-oxo-2,3-dihydro-1H-imidazo[1,2-a]pyrrolo[3,4-e]pyridine-7-carboxylic acid (8 mg, 0.01 mmol) and HATU (10 mg, 0.03 mmol) in DMF (1 mL) was cooled to 0° C., then DIPEA (10 uL) and methylamine (2M THF solution, 20 uL) were added. The resulting mixture was warmed to rt and stirred for 1 h, then quenched with water. The mixture was then extracted with EtOAc. The combined organics were dried with anhydrous sodium sulfate, filtered, and concentrated under reduced pressure. The residue was then dissolved in 20% TfOH in TFA (1 mL), and heated to 70° C. for 10 min. The reaction mixture was cooled to room temperature and concentrated. The residue was dissolved in methanol and purified by prep-HPLC (column: Sunfire prep C18 column, 30*150 mm, 5 μm; mobile phase A: water (0.1% TFA), mobile phase B: acetonitrile; flow rate: 60 mL/min); eluted fractions were collected and lyophilized to provide the TFA salt of the desired product as a white solid. LCMS calculated for C₂₅H₁₆ClF₅N₅O₃ (M+H)⁺ m/z=564.1; found 564.1.

### Example 8-15

Examples 8-15 of Table 2 were prepared similarly as described for Example 7, using the corresponding amines instead of methylamine.

TABLE 2

| | | LC-MS found |
|---|---|---|
| Example # | Structure and Chemical Name | m/z (M + H )⁺ |
| 8 | 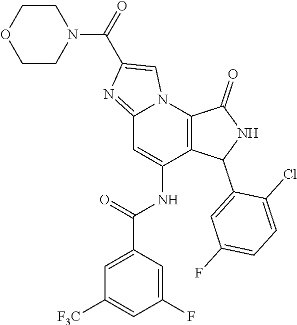 [SMILES: O=C(Nc1cc2nc(C(=O)N3CCOCC3)cn2c2c1C(c1cc(F)ccc1Cl)NC2=O)c1cc(F)cc(C(F)(F)F)c1] | 620.1 |
| | N-(3-(2-chloro-5-fluorophenyl)-7-(morpholine- | |
| | 4-carbonyl)-1-oxo-2,3-dihydro-1H-imidazo[1,2- | |
| | a]pyrrolo[3,4-e]pyridin-4-yl)-3-fluoro-5- | |
| | (trifluoromethyl)benzamide | |
| 9 | 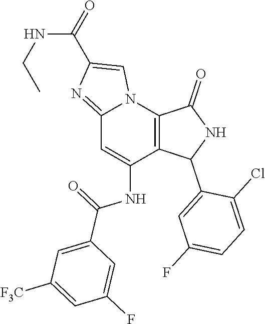 [SMILES: CCNC(=O)c1cn2c3c(c(NC(=O)c4cc(F)cc(C(F)(F)F)c4)cc2n1)C(c1cc(F)ccc1Cl)NC3=O] | 578.1 |
| | 3-(2-chloro-5-fluorophenyl)-N-ethyl-4-(3- | |
| | fluoro-5-(trifluoromethyl)benzamido)-1-oxo- | |
| | 2,3-dihydro-1H-imidazo[1,2-a]pyrrolo[3,4- | |
| | elpyridine-7-carboxamide | |
| 10 | 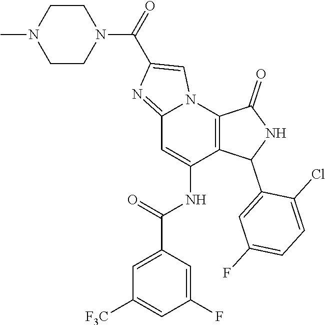 [SMILES: CN1CCN(C(=O)c2cn3c4c(c(NC(=O)c5cc(F)cc(C(F)(F)F)c5)cc3n2)C(c2cc(F)ccc2Cl)NC4=O)CC1] | 633.1 |
| | N-(3-(2-chloro-5-fluorophenyl)-7-(4- | |
| | methylpiperazine-1-carbonyl)-1-oxo-2,3- | |
| | dihydro-1H-imidazo[1,2-a]pyrrolo[3,4- | |
| | e]pyridin-4-y1)-3-fluoro-5- | |
| | (trifluoromethyl)benzamide | |
| 11 | 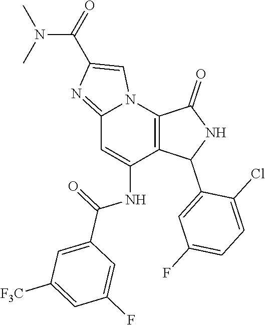 [SMILES: CN(C)C(=O)c1cn2c3c(c(NC(=O)c4cc(F)cc(C(F)(F)F)c4)cc2n1)C(c1cc(F)ccc1Cl)NC3=O] | 578.1 |
| | 3-(2-chloro-5-fluorophenyl)-4-(3-fluoro-5- | |
| | (trifluoromethyl)benzamido)-N,N-dimethyl-1- | |
| | oxo-2,3-dihydro-1H-imidazo[1,2-a]pyrrolo[3,4- | |
| | e]pyridine-7-carboxamide | |
| 12 | 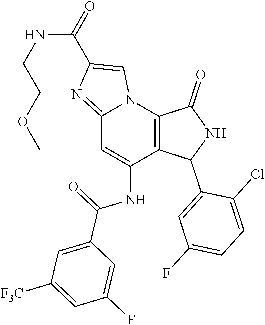 [SMILES: COCCNC(=O)c1cn2c3c(c(NC(=O)c4cc(F)cc(C(F)(F)F)c4)cc2n1)C(c1cc(F)ccc1Cl)NC3=O] | 608.1 |
| | 3-(2-chloro-5-fluorophenyl)-4-(3-fluoro-5- | |
| | (trifluoromethyl)benzamido)-N-(2- | |
| | methoxyethyl)-1-oxo-2,3-dihydro-1H-imidazo[1,2- | |
| | a]pyrrolo[3,4-e]pyridine-7-carboxamide | |
| 13 | 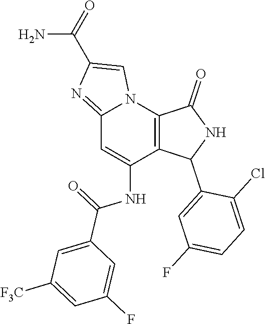 [SMILES: NC(=O)c1cn2c3c(c(NC(=O)c4cc(F)cc(C(F)(F)F)c4)cc2n1)C(c1cc(F)ccc1Cl)NC3=O] | 550.1 |
| | 3-(2-chloro-5-fluorophenyl)-4-(3-fluoro-5- | |
| | (trifluoromethyl)benzamido)-1-oxo-2,3- | |
| | dihydro-1H-imidazo[1,2-a]pyrrolo[3,4- | |
| | elpyridine-7-carboxamide | |
| 14 | 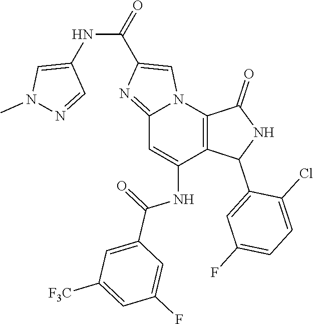 [SMILES: Cn1cc(NC(=O)c2cn3c4c(c(NC(=O)c5cc(F)cc(C(F)(F)F)c5)cc3n2)C(c2cc(F)ccc2Cl)NC4=O)cn1] | 630.1 |
| | 3-(2-chloro-5-fluorophenyl)-4-(3-fluoro-5- | |
| | (trifluoromethyl)benzamido)-N-(1-methyl-1H- | |
| | pyrazol-4-yl)-1-oxo-2,3-dihydro-1H-imidazo[1,2- | |
| | a]pyrrolo[3,4-e]pyridine-7-carboxamide | |
| 15 | 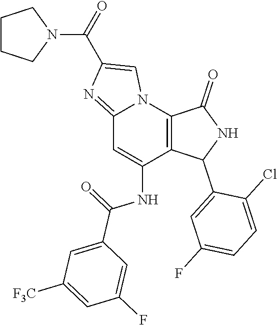 [SMILES: O=C(Nc1cc2nc(C(=O)N3CCCC3)cn2c2c1C(c1cc(F)ccc1Cl)NC2=O)c1cc(F)cc(C(F)(F)F)c1] | 604.1 |
| | N-(3-(2-chloro-5-fluorophenyl)-1-oxo-7- | |
| | (pyrrolidine-1-carbonyl)-2,3-dihydro-1H- | |
| | imidazo[1,2-a]pyrrolo[3,4-e]pyridin-4-yl)-3- | |
| | fluoro-5-(trifluoromethyl)benzamide | |

pS473 AKT Assay Protocol

SKBR3 (PIK3CA WT), MCF7 (PIK3CA E545K), and T47D (PIK3CA H1047R) cells were cultured using 10-cm petri dishes with recommended medium plus 10% fetal bovine serum. One day before the assay, cells were seeded in 96-well plates at a final density of 10,000 cells per well. After overnight incubation in complete medium, cells were treated with different concentrations of PI3Kα inhibitors for 2 h. Cells were then fixed using 4% paraformaldehyde at room temperature for 20 min. Aspirate 4% paraformaldehyde, and wash cells using 1× regular phosphate buffered saline 3 times, 5 min each. Aspirate any residual phosphate buffered saline and block cells using 10% goat serum containing 1% bovine serum albumin and 0.3% Triton X-100 at room temperature for 1 h. Without any additional washing, primary antibodies (rabbit anti-pSer473 AKT) were diluted using blocking buffer and added at a final volume of 50 microliter per well. Keep assay plates with primary antibodies overnight at 4° C. Wash cells using 1× regular phosphate buffered saline 3 times, 5 min each. After the final wash, incubate cells with horseradish peroxidase-conjugated secondary antibodies (goat Anti-rabbit IgG) diluted using the same blocking buffer at room temperature for 1 h. Wash cells thoroughly using 1× regular phosphate buffered saline 3 times, 5 min each. Aspirate any residual phosphate buffered saline. Add Super-Signal ELISA Pico Chemiluminescent Substrate at a final volume of 100 microliter per well. Read plates on i3x Multi-Mode Microplate Reader and calculate IC₅₀ values using GraphPad Prism software.

Results of the assay described above are presented in Table 3. Compounds denoted of the present disclosure showed IC₅₀ values in the following ranges:

$${IC}_{50} \leq 500 nM ; \qquad A$$

$$500 nM < {IC}_{50} \leq 1000 nM ; \qquad B$$

$$1000 nM < {IC}_{50} \leq 5000 nM ; \qquad C$$

$$5000 nM < {IC}_{50} \leq 10 , 000 nM . \qquad D$$

$$10 , 000 nM < {IC}_{50} \qquad E$$

TABLE 3

| | SKBR3_IC₅₀ | MCF7_IC₅₀ | T47D_IC₅₀ |
|---|---|---|---|
| Compound | (nM) | (nM) | (nM) |
| I-1 | C | A | A |
| I-2 | B | A | A |
| I-3 | B | A | A |
| I-4 | A | A | A |
| I-5 | C | B | A |
| I-6 | A | A | A |
| 7 | | | A |
| 8 | | | A |
| 9 | | | A |
| 10 | | | C |
| 11 | | | A |
| 12 | | | A |
| 13 | | | A |
| 14 | | | A |
| 15 | | | A |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula I: 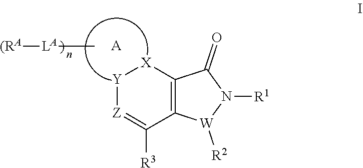
or a pharmaceutically acceptable salt thereof, wherein:
X is N and Y is C; or X is C and Y is N;
Ring A is 5- to 7-membered saturated or partially unsaturated monocyclic heterocyclyl having 1-3 heteroatoms independently selected from nitrogen, oxygen, and sulfur, or 5- to 6-membered monocyclic heteroaryl having 1-4 heteroatoms independently selected from nitrogen, oxygen, and sulfur;
each L^{A} is independently a covalent bond or optionally substituted bivalent C₁₋₆ aliphatic;
each R^{A} is independently oxo, halogen, —CN, —OR^{A1}, —SR^{A1}, —N(R^{A1})₂, —NO₂, —C(O)R^{A1}, —C(O)OR^{A1}, —C(O)N(R^{A1})₂, —C(O)NR^{A1}(OR^{A1}), —OC(O)R^{A1}, —OC(O)N(R^{A1})₂, —OC(O)OR^{A1}, —OSO₂R^{A1}, —OSO₂N(R^{A1})₂, —N(R^{A1})C(O)R^{A1}, —NR^{A1}C(O)OR^{A1}, —NR^{A1}C(O)N(R^{A1})₂, —N(R^{A1})SO₂R^{A1}, —NR^{A1}S(O)₂N(R^{A1})₂, —NR^{A1}OR^{A1}, —NR^{A1}S(O)R^{A1}, —NR^{A1}S(O)N(R^{A1})₂, —S(O)R^{A1}, —SO₂R^{A1}, —S(O)N(R^{A1})₂, —SO₂N(R^{A1})₂, —SO₃R^{A1}, —C(═NR^{m})R^{A1}, —C(═NR^{m})N(R^{A1})₂, —NR^{A1}C(═NR^{m})R^{A1}, —NR^{A1}C(═NR^{m})N(R^{A1})₂, —NR^{A1}S(O)(═NR^{m})R^{A1}, —NR^{A1}S(O)(═NR^{m})N(R^{A1})₂, —OS(O)(═NR^{m})R^{A1}, —S(O)(═NR^{m})R^{A1}, —S(O)(═NR^{m})N(R^{A1})₂, —P(O)(R^{A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A} is independently optionally substituted with 1, 2, 3, 4, 5, or 6 R^{AG} substituents;
R¹ is hydrogen or an optionally substituted group selected from C₁₋₆ aliphatic, 3- to 7-membered saturated or partially unsaturated monocyclic carbocyclyl, phenyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur;
W is CR^{W} or N;
R^{W} is hydrogen or -L^{W}-R^{2G};
L^{W} is a covalent bond or optionally substituted bivalent C₁₋₆ aliphatic;
R² is C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R² is independently optionally substituted with 1, 2, 3, 4, 5, or 6 R^{2G} substituents; or
R² and R^{W} are taken together with the carbon atom to which they are attached to form an optionally substituted ring selected from 3- to 7-membered saturated or partially unsaturated monocyclic carbocyclyl, 5- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 5- to 10-membered saturated or partially unsaturated bicyclic heterocyclyl having 1-4 heteroatoms independently selected from nitrogen, oxygen, and sulfur;
R³ is -L³-R^{3A};
L³ is a covalent bond or optionally substituted bivalent C₁₋₆ aliphatic;
R^{3A} is hydrogen, halogen, —CN, —OR^{3A1}, —SR^{3A1}, —N(R^{3A1})₂, —NO₂, —C(O)R^{3A1}, —C(O)OR^{3A1}, —C(O)N(R^{3A1})₂, —C(O)NR^{3A1}(OR^{3A1}), —OC(O)R^{3A1}, —OC(O)N(R^{3A1})₂, —OC(O)OR^{3A1}, —OSO₂R^{3A1}, —OSO₂N(R^{3A1})₂, —N(R^{3A1})C(O)R^{3A1}, —NR^{3A1}C(O)OR^{3A1}, —NR^{3A1}C(O)N(R^{3A1})₂, —N(R^{3A1})SO₂R^{3A1}, —NR^{3A1}S(O)₂N(R^{3A1})₂, —NR^{3A1}OR^{3A1}, —NR^{3A1}S(O)R^{3A1}, —NR^{3A1}S(O)N(R^{3A1})₂, —S(O)R^{3A1}, —SO₂R^{3A1}, —S(O)N(R^{3A1})₂, —SO₂N(R^{3A1})₂, —SO₃R^{3A1}, —C(═NR^{m})R^{3A1}, —C(═NR^{m})N(R^{3A1})₂, —NR^{3A1}C(═NR^{m})R^{3A1}, —NR^{3A1}C(═NR^{m})N(R^{3A1})₂, —NR^{3A1}S(O)(═NR^{m})R^{3A1}, —NR^{3A1}S(O)(═NR^{m})N(R^{3A1})₂, —OS(O)(═NR^{m})R^{3A1}, —S(O)(═NR^{m})R^{3A1}, —S(O)(═NR^{m})N(R^{3A1})₂, —P(O)(R^{3A1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{3A} is independently optionally substituted with 1, 2, 3, 4, 5, or 6 R^{3AG} substituents;
Z is N or CR^{Z};
R^{Z} is -L^{Z}-R^{ZA};
L^{Z} is a covalent bond or optionally substituted bivalent C₁₋₆ aliphatic;
R^{ZA} is hydrogen, halogen, —CN, —OR^{ZA1}, —SR^{ZA1}, —N(R^{ZA1})₂, —NO₂, —C(O)R^{ZA1}, —C(O)OR^{ZA1}, —C(O)N(R^{ZA1})₂, —C(O)NR^{ZA1}(OR^{ZA1}), —OC(O)R^{ZA1}, —OC(O)N(R^{ZA1})₂, —OC(O)OR^{ZA1}, —OSO₂R^{ZA1}, —OSO₂N(R^{ZA1})₂, —N(R^{ZA1})C(O)R^{ZA1}, —NR^{ZA1}C(O)OR^{ZA1}, —NR^{ZA1}C(O)N(R^{ZA1})₂, —N(R^{ZA1})SO₂R^{ZA1}, —NR^{ZA1}S(O)₂N(R^{ZA1})₂, —NR^{ZA1}OR^{ZA1}, —NR^{ZA1}S(O)R^{ZA1}′, —NR^{ZA1}S(O)N(R^{ZA1})₂, —S(O)R^{ZA1}, —SO₂R^{ZA1}, —S(O)N(R^{ZA1})₂, —SO₂N(R^{ZA1})₂, —SO₃R^{ZA1}, —C(═NR^{m})R^{ZA1}, —C(═NR^{m})N(R^{ZA1})₂, —NR^{ZA1}C(═NR^{m})R^{ZA1}, —NR^{ZA1}C(═NR^{m})N(R^{ZA1})₂, —NR^{ZA1}S(O)(═NR^{m})R^{ZA1}, —NR^{ZA1}S(O)(═NR^{m})N(R^{ZA1})₂, —OS(O)(═NR^{m})R^{ZA1}, —S(O)(═NR^{m})R^{ZA1}, —S(O)(═NR^{m})N(R^{ZA1})₂, —P(O)(R^{ZA1})₂, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{ZA} is independently optionally substituted with 1, 2, 3, 4, 5, or 6 R^{ZAC} substituents;
R^{A1}, R^{3A1}, and R^{ZA1} are each independently hydrogen, C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur,
wherein each of the C₁₋₆ aliphatic, 3- to 7-membered monocyclic carbocyclyl, 5- to 10-membered bicyclic carbocyclyl, phenyl, 8- to 10-membered bicyclic aryl, 3- to 7-membered monocyclic heterocyclyl, 5- to 10-membered bicyclic heterocyclyl, 5- to 6-membered monocyclic heteroaryl, and 8- to 10-membered bicyclic heteroaryl of R^{A1}, R^{3A1}, or R^{ZA1} is independently optionally substituted with 1, 2, 3, 4, 5, or 6 R^{G1} substituents;
or two R^{A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
or two R^{3A1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
or two R^{ZA1} when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
R^{AG}, R^{2G}, R^{3AG}, R^{ZAG}, and R^{G1} are each independently hydrogen, halogen, —CN, —OR, —SR, —N(R)₂, —NO₂, —C(O)R, —C(O)OR, —C(O)N(R)₂, —C(O)NR(OR), —OC(O)R, —OC(O)N(R)₂, —OC(O)OR, —OSO₂R, —OSO₂N(R)₂, —N(R)C(O)R, —NRC(O)OR, —NRC(O)N(R)₂, —N(R)SO₂R, —NRS(O)₂N(R)₂, —NROR, —NRS(O)R, —NRS(O)N(R)₂, —S(O)R, —SO₂R, —S(O)N(R)₂, —SO₂N(R)₂, —SO₃R, —C(═NR^{m})R, —C(═NR^{m})N(R)₂, —NRC(═NR^{m})R, —NRC(═NR^{m})N(R)₂, —NRS(O)(═NR^{m})R, —NRS(O)(═NR^{m})N(R)₂, —OS(O)(═NR^{m})R, —S(O)(═NR^{m})R, —S(O)(═NR^{m})N(R)₂, —P(O)(R)₂, or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
each R is independently hydrogen or an optionally substituted group selected from C₁₋₆ aliphatic, 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or
two R when attached to the same nitrogen atom are taken together to form an optionally substituted ring selected from 3- to 7-membered saturated or partially unsaturated monocyclic heterocyclyl having 0-2 additional heteroatoms independently selected from nitrogen, oxygen, and sulfur, and 5- to 10-membered saturated or partially unsaturated bicyclic heterocyclyl having 0-3 additional heteroatoms independently selected from nitrogen, oxygen, and sulfur;
each R^{m} is independently —OH, —CN, or R; and
n is 0, 1, 2, 3, 4, or 5.

2. The compound of claim 1, wherein the compound is of Formula II: 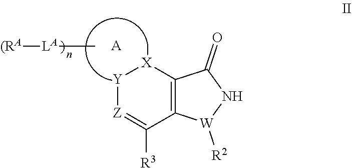
or a pharmaceutically acceptable salt thereof.

3. The compound of claim 1, wherein the compound is of Formula III: 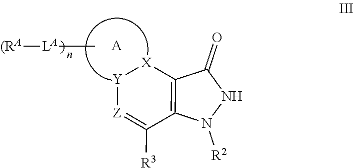
or a pharmaceutically acceptable salt thereof.

4. The compound of claim 1, wherein the compound is of Formula IV: 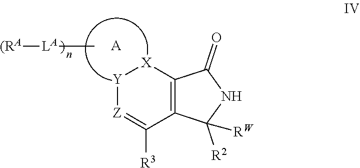
or a pharmaceutically acceptable salt thereof.

5. The compound of claim 1, or a pharmaceutically acceptable salt thereof, wherein X is C and Y is N.

6. The compound of claim 1, or a pharmaceutically acceptable salt thereof, wherein X is N and Y is C.

7. The compound of claim 1, or a pharmaceutically acceptable salt thereof, wherein Ring A is 5- to 6-membered monocyclic heteroaryl having 1-4 heteroatoms independently selected from nitrogen, oxygen, and sulfur.

8. The compound of claim 1, or a pharmaceutically acceptable salt thereof, wherein Ring A is 5-membered monocyclic heteroaryl having 1-4 heteroatoms nitrogen atoms.

9. The compound of claim 1, or a pharmaceutically acceptable salt thereof, wherein L^{A} is a covalent bond.

10. The compound of claim 1, or a pharmaceutically acceptable salt thereof, wherein R^{A} is —C(O)OR^{A1}.

11. The compound of claim 1, or a pharmaceutically acceptable salt thereof, wherein each R^{A1} is independently C₁₋₆ aliphatic.

12. The compound of claim 1, or a pharmaceutically acceptable salt thereof, wherein 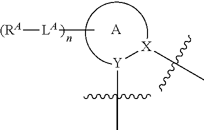
is selected from the group consisting of

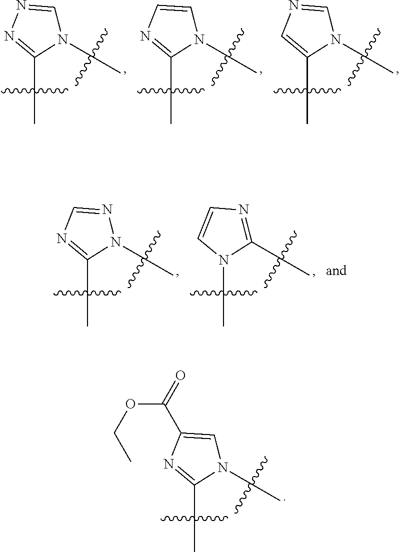 [SMILES: *c1nncn1* ; *c1nccn1* ; *c1cncn1* ; *c1ncnn1* ; *c1nccn1* ; *c1nc(C(=O)OCC)cn1*]

13. The compound of claim 1, or a pharmaceutically acceptable salt thereof, wherein R^{W} is hydrogen.

14. The compound of claim 1, or a pharmaceutically acceptable salt thereof, wherein R² is phenyl, 8- to 10-membered bicyclic aryl, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, each of which is optionally substituted with 1, 2, 3, 4, 5, or 6 independently selected R^{2G} substituents.

15. The compound of claim 1, or a pharmaceutically acceptable salt thereof, wherein R² is phenyl optionally substituted with 1, 2, 3, 4, 5, or 6 R^{2G} substituents.

16. The compound of claim 1, or a pharmaceutically acceptable salt thereof, wherein each R^{2G} is independently halogen.

17. The compound of claim 1, or a pharmaceutically acceptable salt thereof, wherein R² is

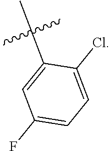 [SMILES: *c1cc(F)ccc1Cl]

18. The compound of claim 1, or a pharmaceutically acceptable salt thereof, wherein L³ is a covalent bond.

19. The compound of claim 1, or a pharmaceutically acceptable salt thereof, wherein R^{3A} is —N(R^{3A1})C(O)R^{3A1} or —NR^{3A1}C(O)N(R^{3A1})₂.

20. The compound of claim 1, or a pharmaceutically acceptable salt thereof, wherein R^{3A} is —N(R^{3A1})C(O)R^{3A1}.

21. The compound of claim 1, or a pharmaceutically acceptable salt thereof, wherein R^{3A} is —NHC(O)R^{3A1} or —NHC(O)N(R^{3A1})₂.

22. The compound of claim 1, or a pharmaceutically acceptable salt thereof, wherein R^{3A} is —NHC(O)R^{3A1}.

23. The compound of claim 1, or a pharmaceutically acceptable salt thereof, wherein R^{3A1} is hydrogen or 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, wherein each of carbocyclyl, phenyl, heterocyclyl, and heteroaryl is independently optionally substituted with 1, 2, 3, 4, 5, or 6 R^{G1} substituents.

24. The compound of claim 1, or a pharmaceutically acceptable salt thereof, wherein R^{3A1} is hydrogen.

25. The compound of claim 1, or a pharmaceutically acceptable salt thereof, wherein R^{3A1} is 3- to 7-membered saturated or partially unsaturated monocyclic carbocyclyl, 5- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 5- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, or 8- to 10-membered bicyclic heteroaryl having 1-4 heteroatoms independently selected from nitrogen, oxygen, and sulfur, each of which is independently optionally substituted with 1, 2, 3, 4, 5, or 6 R^{G}1 substituents.

26. The compound of claim 1, or a pharmaceutically acceptable salt thereof, wherein R³ is

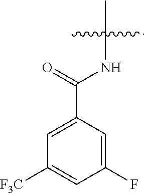 [SMILES: *NC(=O)c1cc(F)cc(C(F)(F)F)c1]

27. The compound of claim 1, or a pharmaceutically acceptable salt thereof, wherein L^{Z} is a covalent bond.

28. The compound of claim 1, or a pharmaceutically acceptable salt thereof, wherein R^{ZA} is hydrogen.

29. The compound of claim 1, or a pharmaceutically acceptable salt thereof, wherein each R^{G1} is independently halogen or optionally substituted C₁₋₆ aliphatic.

30. The compound of claim 1, or a pharmaceutically acceptable salt thereof, wherein n is 0 or 1.

31. The compound of claim 1, wherein the compound is selected from: 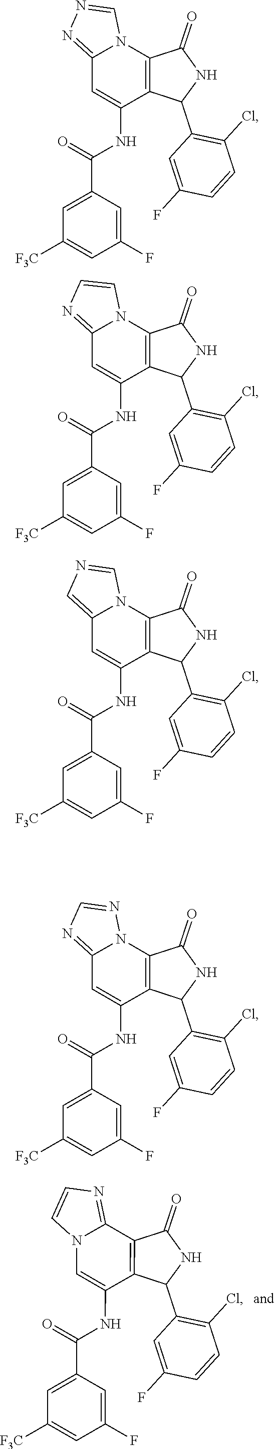 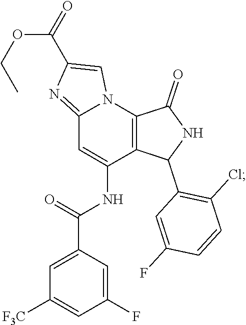 [SMILES: CCOC(=O)c1cn2c3c(c(NC(=O)c4cc(F)cc(C(F)(F)F)c4)cc2n1)C(c1cc(F)ccc1Cl)NC3=O]
or a pharmaceutically acceptable salt thereof.

32. The compound of claim 1, wherein the compound is selected from: 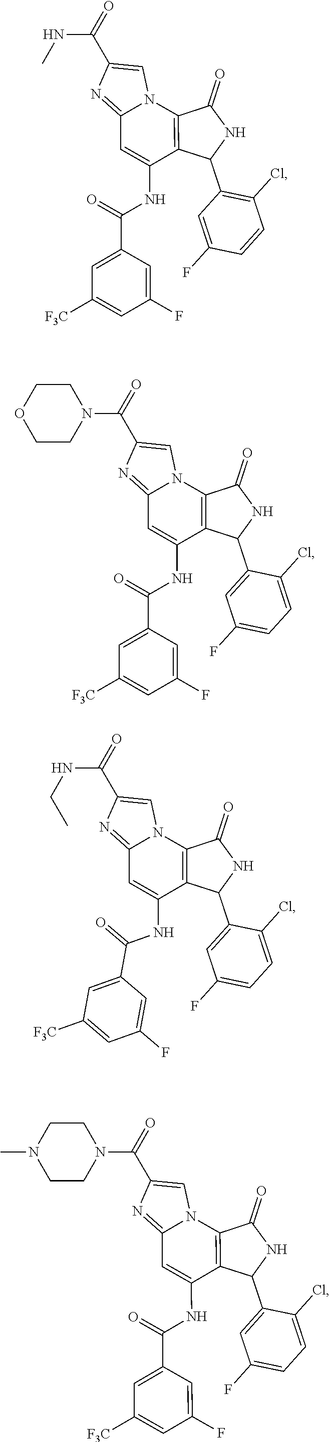 [SMILES: CNC(=O)c1cn2c3c(c(NC(=O)c4cc(F)cc(C(F)(F)F)c4)cc2n1)C(c1cc(F)ccc1Cl)NC3=O ; O=C(Nc1cc2nc(C(=O)N3CCOCC3)cn2c2c1C(c1cc(F)ccc1Cl)NC2=O)c1cc(F)cc(C(F)(F)F)c1 ; CCNC(=O)c1cn2c3c(c(NC(=O)c4cc(F)cc(C(F)(F)F)c4)cc2n1)C(c1cc(F)ccc1Cl)NC3=O ; CN1CCN(C(=O)c2cn3c4c(c(NC(=O)c5cc(F)cc(C(F)(F)F)c5)cc3n2)C(c2cc(F)ccc2Cl)NC4=O)CC1] 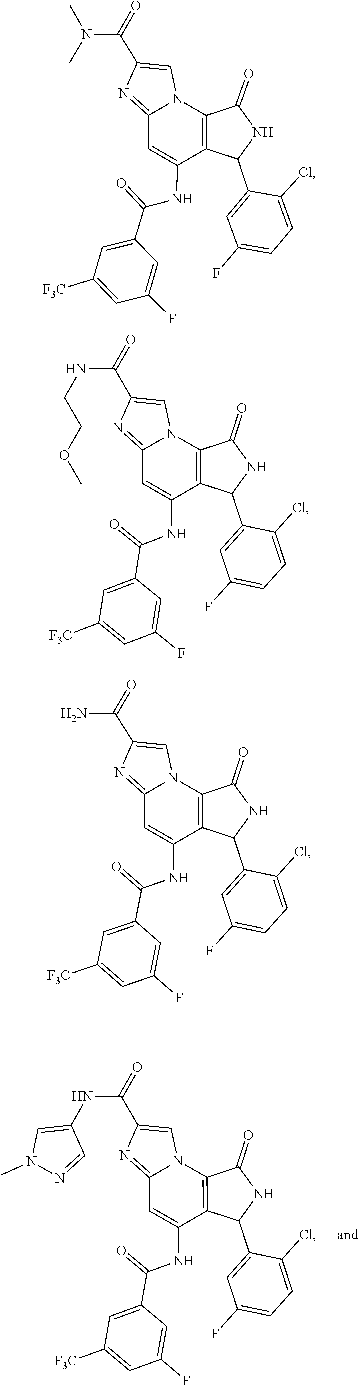 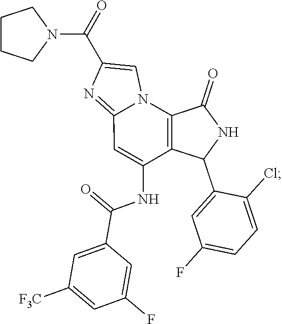 [SMILES: O=C(Nc1cc2nc(C(=O)N3CCCC3)cn2c2c1C(c1cc(F)ccc1Cl)NC2=O)c1cc(F)cc(C(F)(F)F)c1]
or a pharmaceutically acceptable salt thereof.

33. A pharmaceutical composition comprising a compound of claim 1, or a pharmaceutically acceptable salt thereof, and a pharmaceutically acceptable carrier.

34. A method of inhibiting PI3Kα, comprising administering to a subject a compound of claim 1, or a pharmaceutically acceptable salt thereof.

35. The method of claim 34, wherein the method selectively inhibits PI3Kα over other PI3K isoforms.

36. The method of claim 34, wherein the method selectively inhibits mutant PI3Kα over wide-type PI3Kα.

37. A method of treating a disease, disorder, or condition associated with PI3Kα, comprising administering to a subject in need thereof a compound of claim 1, or a pharmaceutically acceptable salt thereof.

38. The method of claim 37, wherein the disease, disorder, or condition is associated with mutant PI3Kα.

39. The method of claim 37, wherein the disease or disorder is CLOVES syndrome (congenital lipomatous overgrowth, vascular malformations, epidermal naevi, scoliosis/skeletal and spinal syndrome), or PIK3CA-related overgrowth syndrome (PROS).

40. A method of treating cancer, comprising administering to a subject in need thereof a compound of claim 1, or a pharmaceutically acceptable salt thereof.

41. The method of claim 40, wherein the cancer is selected from breast cancer, brain cancer, prostate cancer, endometrial cancer, gastric cancer, leukemia, lymphoma, sarcoma, colorectal cancer, lung cancer, ovarian cancer, skin cancer, and head and neck cancer.
